(19) United States
(12) Patent Application Publication
(10) Pub. No.: US 2021/0163420 A1
(43) Pub. Date: Jun. 3, 2021

(54) Novel Spiro and Cyclic Bis-Benzylidine Proteasome Inhibitor for the Treatment of Cancer, Diabetes and Neurological Disorders

(71) Applicant: UP THERAPEUTICS, INC., Elkridge, MD (US)

(72) Inventor: Yung-Nien Chang, Elkridge, MD (US)

(73) Assignee: UP THERAPEUTICS, INC., Elkridge, MD (US)

(21) Appl. No.: 17/154,306

(22) Filed: Jan. 21, 2021

Related U.S. Application Data
(63) Continuation-in-part of application No. 16/481,562, filed on Jul. 29, 2019, now Pat. No. 10,947,199.
(60) Provisional application No. 62/451,920, filed on Jan. 30, 2017.

(51) Int. Cl. C07D 221/20; A61P 35/00

(52) U.S. Cl. CPC C07D 221/20; A61P 35/00

(57) ABSTRACT

Described herein are spiro and cyclic bis-benzylidine proteasome inhibitors, which inhibit the proteasome function through either ubiquitin receptor ADRM1/RPN13 or proteasome DUB enzymes (USP14, UCH37 and RPN11), and which can be used for the treatment of cancers/diabetes/neurological disorders.

### CROSS-REFERENCE TO RELATED APPLICATIONS

This application is a Continuation-in-Part of U.S. patent application Ser. No. 16/481,562, filed Jul. 29, 2019, which was a National Stage Entry of PCT/US2018/015826, filed Jan. 30, 2018, which claims priority from U.S. Provisional Patent Application No. 62/451,920, filed Jan. 30, 2017. The contents of these priority applications are incorporated herein by reference in their entirety.

### BACKGROUND

Ubiquitin-Proteasome System (UPS) play a vital role in cellular homeostasis, cell cycle progression and signaling pathways that are altered in many diseases. Modulating UPS function with small molecules that directly bind to the proteasomal proteins can be useful to treat wide variety of diseases specifically cancers, diabetes and neurological disorders. Targeting the aberrant metabolism of cancer cells is an emerging approach for cancer therapy. Indeed, despite early skepticism that inhibiting protein degradation with a proteasome inhibitor would provide a sufficient therapeutic index for cancer therapy, over the last decade three proteasorne inhibitors that target the catalytic function of the 20S proteasorne were approved by FDA to treat multiple myeloma and mantle cell lymphoma. However, their limited efficacy against solid tumors, toxicities and the emergence of resistant multiple myeloma has driven search for alternative proteasome inhibitors with distinct and complementary mechanisms of action.

### SUMMARY

Provided herein are spiro and cyclic bis-benzylidine small molecules (Up I, II and III, Up compounds hereafter), a new type of proteasome inhibitor, that inhibit the proteasome function through either ubiquitin receptor ADRM1/RPN13 or proteasome DUB enzymes (USP14, UCH37 and RPN11) can be used for the treatment of cancers/diabetes/neurological disorders. The Up compounds bind to the 19S regulatory particle of the proteasome which triggers a rapid and toxic accumulation of high molecular weight poly-ubiqutinated protein aggregates, reflecting inhibition of deubiquitinase activity and substrate recognition by the proteasome.

### BRIEF DESCRIPTION OF THE DRAWINGS

FIG. 1: Up109 and Up117 stabilized proteasome-targeted firefly luciferase reporter.

FIG. 2: Up109 and Up117 accumulated poly ubiquitin tagged proteins.

FIG. 3: Up109 and Up117 inhibited NFKB signaling.

FIG. 4: Up109 and Up117 binds to RPN13.

FIG. 5A, FIG. 5B, and FIG. 5C: Accumulation of PolyUB proteins with the treatment of compounds: (A) HeLa cells treated with Up compounds (1 μM) for 4 h and immunoblotted with K48 linked anti-Ub antibody; (B) OV2008 cells treated with Up compounds (1 μM) for 12 h and immunoblotted with anti-Ub antibody; (C) LNCaP cells treated with Up compounds for 4 h and immunoblotted with anti-Ub antibody.

FIG. 6: Stabilization of 4UBFL reporter protein with Up109 treatment.

FIG. 7: Up109 binds to RPN13.

FIG. 8A and FIG. 8B: (A) CHOP-10 quantification in ES2 cells treated with Up109 for 12 h; (B) Annexin-V staining by FACS analysis of ES2 cells treated with Up109 and Bortezomib for 12 h.

FIG. 9: BALB/C mice were electroporated with 4UBFL gene and treated with Up109 and the IVIS imaging was used to quantify luciferase activity.

FIG. 10: In vivo efficacy of Up109 against ES2Lu tumor growth.

FIG. 11: Effect of Up284 against the viability of different cancer cell lines treated with Up284 for 72 hours.

FIG. 12A, FIG. 12B and FIG. 12C: Evidence of proteasome inhibition in ES2 (FIG. 12A), HCC1806 (FIG. 12B), and U251-MG (FIG. 12C) cells showing accumulation of high MW polyUb proteins with Up compounds treatment at indicated times and concentrations (μM) in a Western blot using ubiquitin antibody. Bz=bortezomib. Actin or Tubulin used as positive controls.

FIG. 13A, FIG. 13B, and FIG. 13C: Up284 binds to RPN13 much more strongly than RA190. FIG. 13A—ES2 cell lysate pretreated with Up compounds (5 μM) and then labeled with biotinylated RA190 (RA190B 20 μM). FIG. 13B—ES2 cell lysate treated with RA190B and biotinylated Up284 (Up284B) at indicated concentrations and probed for HRP-Streptavidin. FIG. 13C—A2780 cell lysate treated with Up284biotin and RA190 biotin at indicated concentrations.

FIG. 14 shows the effects of Up284 on stabilization of proteasome dependent reporter protein 8Ub-FL.

FIG. 15A, FIG. 15B, FIG. 15C, and FIG. 15D show the induction of ER stress response by Up284. FIG. 15A shows the results of RT-qPCR of U251MG cells showing ER stress response element CHOP-10 increase with Up284 treatment. FIG. 15B Shows the measured bioluminiscence of 293 cells expressing NFkB dependent luciferase, showing reduced NFkB activity with Up284 treatment. with indicated doses of Up284. FIG. 15C shows reduced GSH/GSSG activities in U251MG with Up284 treatment; FIG. 15D shows that increased ROS is seen with Up284 treatment.

FIG. 16A and FIG. 16B show that Up284 treatment cased apoptotic cell death in glioblastoma cell line U251MG. FIG. 16A shows the results of FACS analysis of U251MG cells showing Annexin-V positive cells after 12 hours of Up284 treatment. FIG. 16B shows cleaved caspase after 12 hr of Up284 treatment.

### DETAILED DESCRIPTION

Provided herein are compounds having the structure of formula either I, or II, or III, shown below

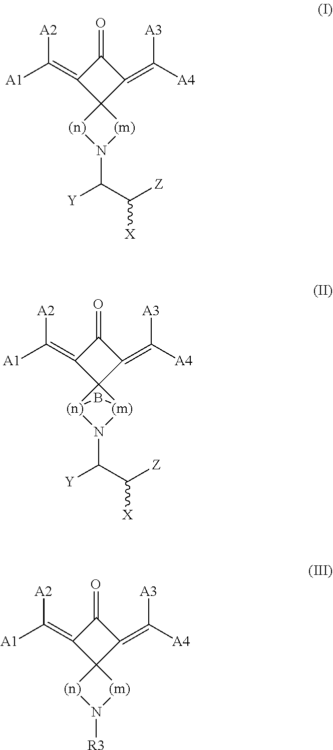

or a pharmaceutically acceptable salt thereof
wherein each of A1, A2, A3, and A4 is one of:
   (i) phenyl, optionally substituted with 1-5 substituents selected from the group consisting of R1, OR1, NR1R2, S(O)qR1, SO₂NR1R2, NR1SO₂R2, C(O)R1, C(O)OR1, C(O)NR1R2, NR1C(O)R2, NR1C(O)OR2, CF₃, and OCF₃;
   (ii) naphthyl, optionally substituted with 1-5 substituents selected from the consisting of R1, OR1, NR1R2, S(O)qR1, SO₂NR1R2, NR1SO₂R2, C(O)R1, C(O)OR1, C(O)NR1R2, NR1C(O)R2, NR1C(O)OR2, CF₃, and OCF₃;
   (iii) a 5 or 6 membered monocyclic heteroaryl group, having 1-3 heteroatoms selected from the group consisting of O, N, and S, optionally substituted with 1-3 substituents selected from the group consisting of R1, OR1, NR1R2, S(O)qR1, SO₂NR1R2, NR1SO₂R2, C(O)R1, C(O)OR1, C(O)NR1R2, NR1C(O)R2, NR1C(O)OR2, CF₃, and OCF₃; and
   (iv) an 8 to 10 membered bicyclic heteroalkyl group containing 1-3 heteroatoms selected from the group consisting of O, N, and S; and the second ring is fused to the first ring using 3 to 4 carbon atoms, and the bicyclic hetero aryl group is optionally substituted with 1-3 substituents selected from the group consisting of R1, OR1, NR1R2, S(O)qR1, SO₂NR1R2, NR1SO₂R2, C(O)R1, C(O)OR1, C(O)NR1 R2, NR1 C(O)R2, NR1C(O)OR2, CF₃, and OCF₃;
   (v) any group belongs to R1 or R2
wherein n and m represent a number of atoms ranging from 0-4 (0,1,2,3,4) and can be a Carbon, Nitrogen or Oxygen. In the case of nitrogen, it can be NH, NR1 or NR2;
wherein X is Hydrogen, OR1 or NP, wherein P is selected from the group consisting of R1, C(O)R1, C(O)OR1, C(O)NR1 R2, S—N(R1)COOR1, and S—N(R1), wherein W and Y are independently selected from the group consisting of O, S, NR1 and CR1 R2, and wherein R1 and R2 are selected from the group consisting of —H, —NO2, —OH, —COON, —NH2, halogen, —CN, and C1-C14 linear or branched alkyl groups, that are optionally substituted with 1-3 substituents selected from the group consisting of C1-C 4 linear or branched alkyl, up to perhalo substituted C1-C14 linear or branched alkyl, C1-C4 alkoxy, hydrogen, nitro, hydroxyl, carboxy, amino, C1-C14 alkylamino, C-i-C-n dialkylamino, halogen, and cyano.;
wherein Z is selected from the group consisting of hydrogen; C1 to C14 linear, branched, or cyclic alkyls; alkenyls, phenyl; benzyl, 1-5 substituted benzyl, C1-C6 alkyl-phenyl, wherein the alkyl moiety is optionally substituted with halogen up to perhalo; up to perhalo substituted C1 to C14 linear or branched alkyls; —(CH2)q-K, where K is a 5 or 6 membered monocyclic heterocyclic ring, containing 1 to 4 atoms selected from oxygen, nitrogen and sulfur, which is saturated, partially saturated, or aromatic, or an 8 to 10 membered bicyclic heteroaryl having 1-4 heteroatoms selected from the group consisting of O, N and S, wherein said alkyl moiety is optionally substituted with halogen up to perhalo, and wherein the variable q is an integer ranging from 0 to 4;
wherein B is (i) R1, C(O)R1, C(O)OR1, C(O)NR1R2, S—N(R)COOR1, S—N(R1)COO(B), S(B); and wherein each R1-R2, other than perhalo substituted C1-C14 linear or branched alkyl, is optionally substituted with 1-3 substituents independently selected from the group consisting of C-1-C14 linear or branched alkyl, up to perhalo substituted C1-C14 linear or branched alkyl, C1 -C3 alkoxy, hydroxyl, carboxy, amino, C1-C3 alkylamino, C1-C6 dialkylamino, halogen, cyano; and
where in R₃ is H, C₁₋₆-alkyl, C₂₋₆-alkenyl; C₁₋₃-alkoxy-C₁₋₆-alkyl-; C₁₋₃-alkoxy-C₂₋₆-alkenyl-; aryl-C₀₋₆-alkyl-;heteroaryl-C₀₋₆-alkyl-; heterocyclyl-C₀₋₆-alkyl-; cycloalkyl-C₀₋₆-alkyl-; [symbol] C₁₋₆-alkyl-COOC₁₋₆-alkyl; z,52 C₂₋₆-alkyl-aryloxy; C₁₋₆-alkyl-heteroaryl; C₁₋₆-alkyl-heterocyclyl; C₁₋₆-alkyl-cycloalkyl; C₁₋₆-alkyl-aryl; COR⁴, where R⁴ is selected from: C₁₋₆-alkyl; C₂₋₆-alkenyl; C₁₋₆-alkoxy; C₁₋₃-alkoxy-C₁₋₆-alkyl-; C₁₋₃-alkoxy-C₂₋₆-alkenyl-; aryl-C₀₋₆-alkyl-; heteroaryl-C₀₋₆-alkyl-;heterocyclyl-C₀₋₆-alkyl-;cycloalkyl-C₀₋₆-alkyl-;z,51 C₁₋₆-alkyl-COOC₁₋₆-alkyl; NH_{2;} [symbol] NHC₁₋₆-alkyl; [symbol] N(C₁₋₆-alkyl)₂; [symbol] C₀₋₆-alkyl-aryloxy.

The compounds described herein bind to proteasomal proteins as either DUB inhibitor or proteasome receptor inhibitor.

Also provided herein is a method of inhibiting proteasomes in a mammal by administering an effective amount of the compound disclosed herein to the mammal. As used herein, the term “mammal” includes, for example, humans, dogs, and cats.

Also provided herein are methods of treating a disease in a mammal by administering to the mammal a therapeutically effective dose of a compound as described herein. The disease to be treated may be, for example, cancer, or diabetes, or neurological disorders.

The compounds disclosed herein may be usefully administered alone or in combination with at least one other therapeutic agent or radiation, as can be determined by a medical professional.

Exemplary compounds which meet the requirements described herein include the following:

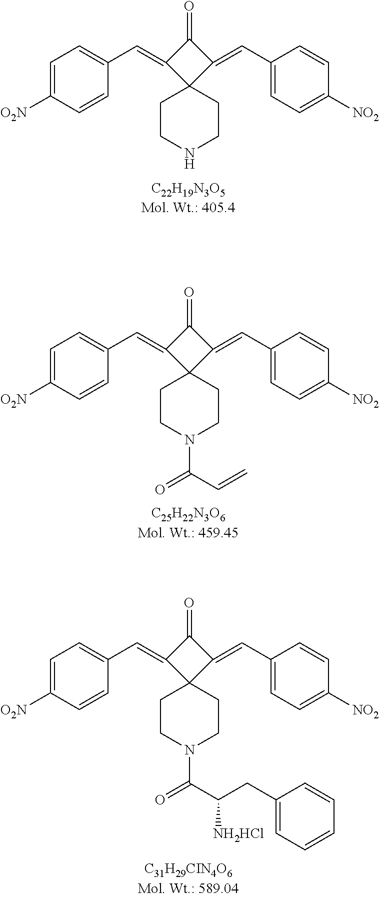

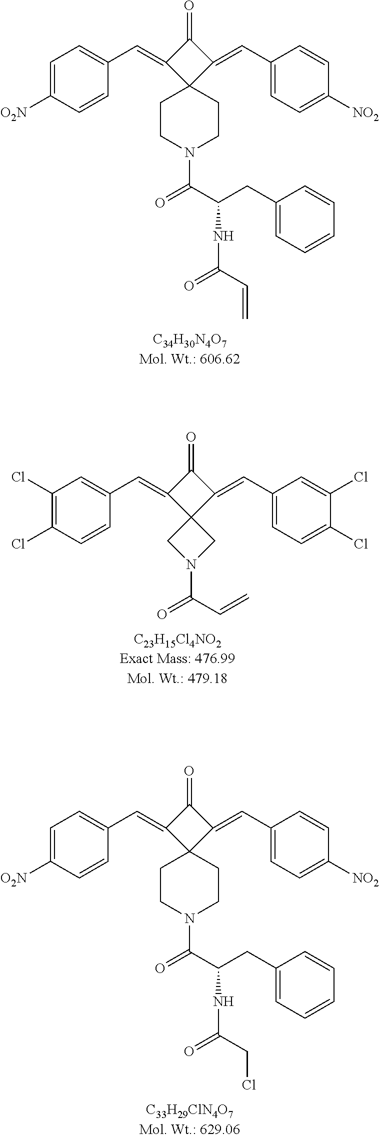 [SMILES: C=CC(=O)N[C@@H](Cc1ccccc1)C(=O)N1CCC2(CC1)/C(=C\c1ccc([N+](=O)[O-])cc1)C(=O)/C2=C/c1ccc([N+](=O)[O-])cc1 ; C=CC(=O)N1CC2(C1)/C(=C\c1ccc(Cl)c(Cl)c1)C(=O)/C2=C/c1ccc(Cl)c(Cl)c1 ; O=C(CCl)N[C@@H](Cc1ccccc1)C(=O)N1CCC2(CC1)/C(=C\c1ccc([N+](=O)[O-])cc1)C(=O)/C2=C/c1ccc([N+](=O)[O-])cc1]

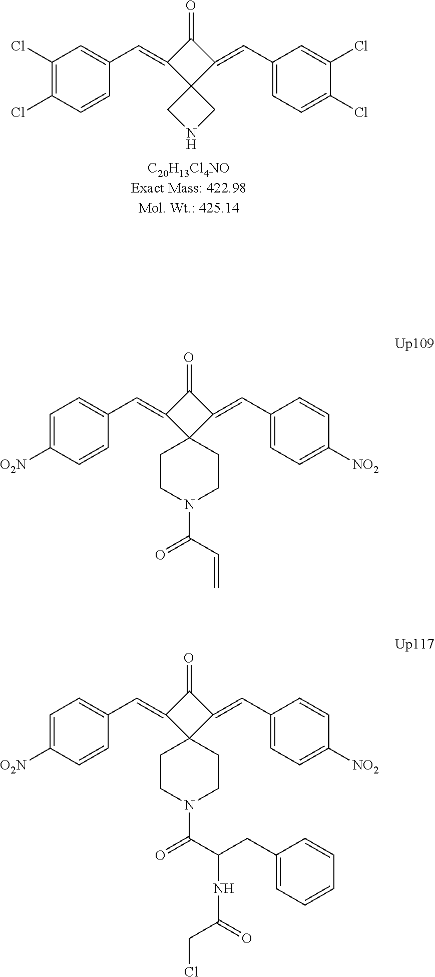 [SMILES: O=C1/C(=C/c2ccc(Cl)c(Cl)c2)C2(CNC2)/C1=C\c1ccc(Cl)c(Cl)c1 ; C=CC(=O)N1CCC2(CC1)/C(=C\c1ccc([N+](=O)[O-])cc1)C(=O)/C2=C/c1ccc([N+](=O)[O-])cc1 ; O=C(CCl)NC(Cc1ccccc1)C(=O)N1CCC2(CC1)/C(=C\c1ccc([N+](=O)[O-])cc1)C(=O)/C2=C/c1ccc([N+](=O)[O-])cc1]

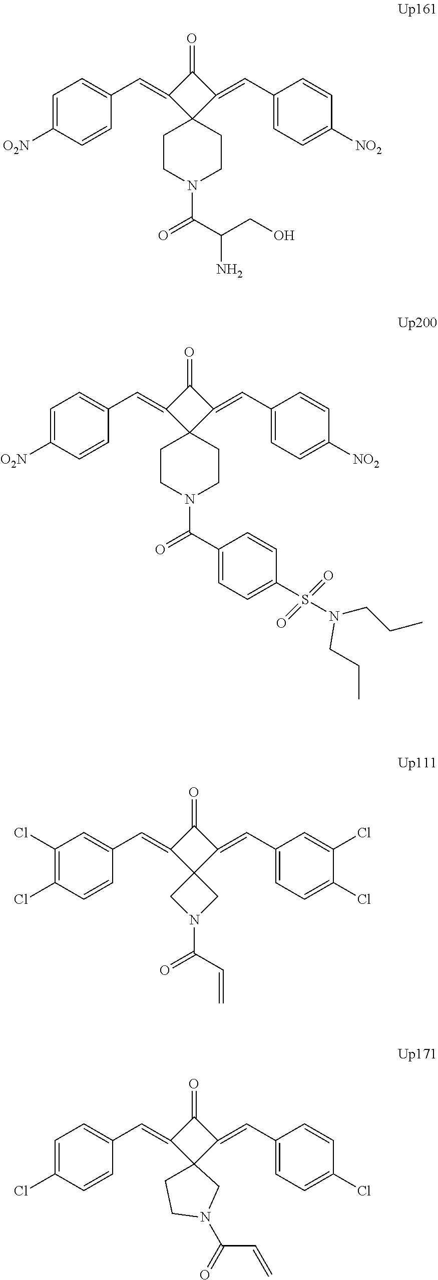 [SMILES: NC(CO)C(=O)N1CCC2(CC1)/C(=C\c1ccc([N+](=O)[O-])cc1)C(=O)/C2=C/c1ccc([N+](=O)[O-])cc1 ; CCCN(CCC)S(=O)(=O)c1ccc(C(=O)N2CCC3(CC2)/C(=C\c2ccc([N+](=O)[O-])cc2)C(=O)/C3=C/c2ccc([N+](=O)[O-])cc2)cc1 ; C=CC(=O)N1CC2(C1)/C(=C\c1ccc(Cl)c(Cl)c1)C(=O)/C2=C/c1ccc(Cl)c(Cl)c1 ; C=CC(=O)N1CCC2(C1)/C(=C\c1ccc(Cl)cc1)C(=O)/C2=C/c1ccc(Cl)cc1]

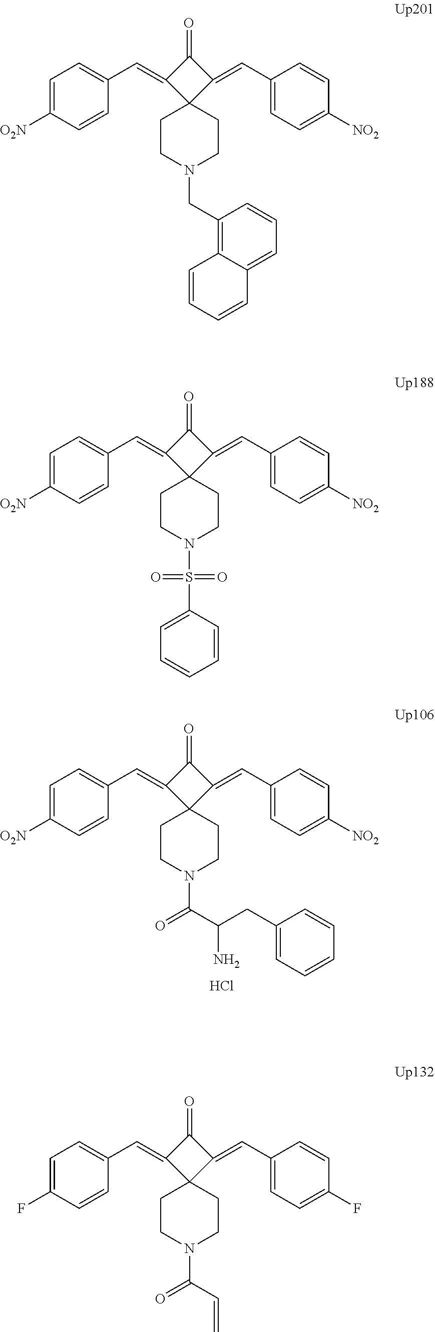 [SMILES: O=C1/C(=C/c2ccc([N+](=O)[O-])cc2)C2(CCN(Cc3cccc4ccccc34)CC2)/C1=C\c1ccc([N+](=O)[O-])cc1 ; O=C1/C(=C/c2ccc([N+](=O)[O-])cc2)C2(CCN(S(=O)(=O)c3ccccc3)CC2)/C1=C\c1ccc([N+](=O)[O-])cc1 ; NC(Cc1ccccc1)C(=O)N1CCC2(CC1)/C(=C\c1ccc([N+](=O)[O-])cc1)C(=O)/C2=C/c1ccc([N+](=O)[O-])cc1 ; Cl ; C=CC(=O)N1CCC2(CC1)/C(=C\c1ccc(F)cc1)C(=O)/C2=C/c1ccc(F)cc1]

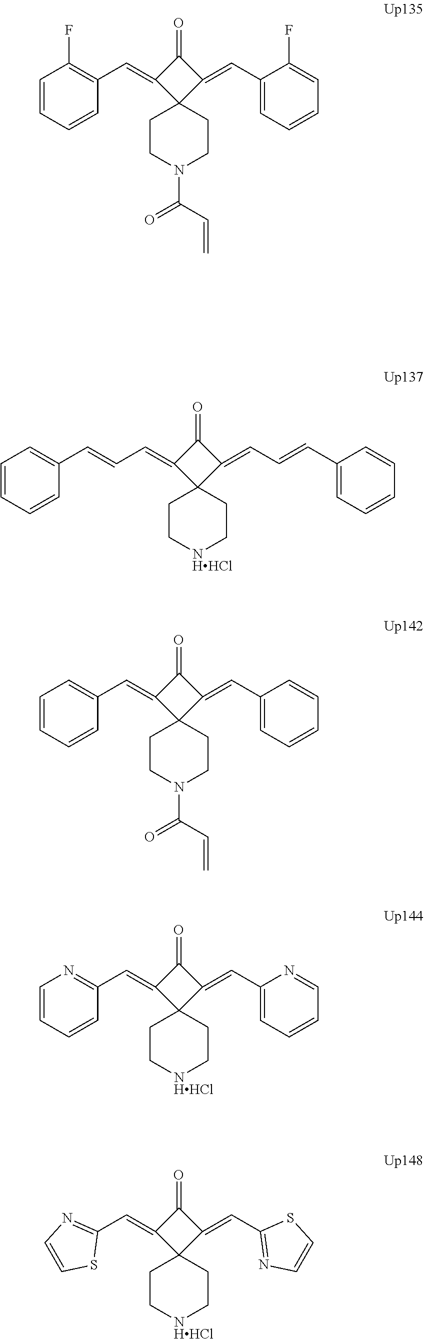

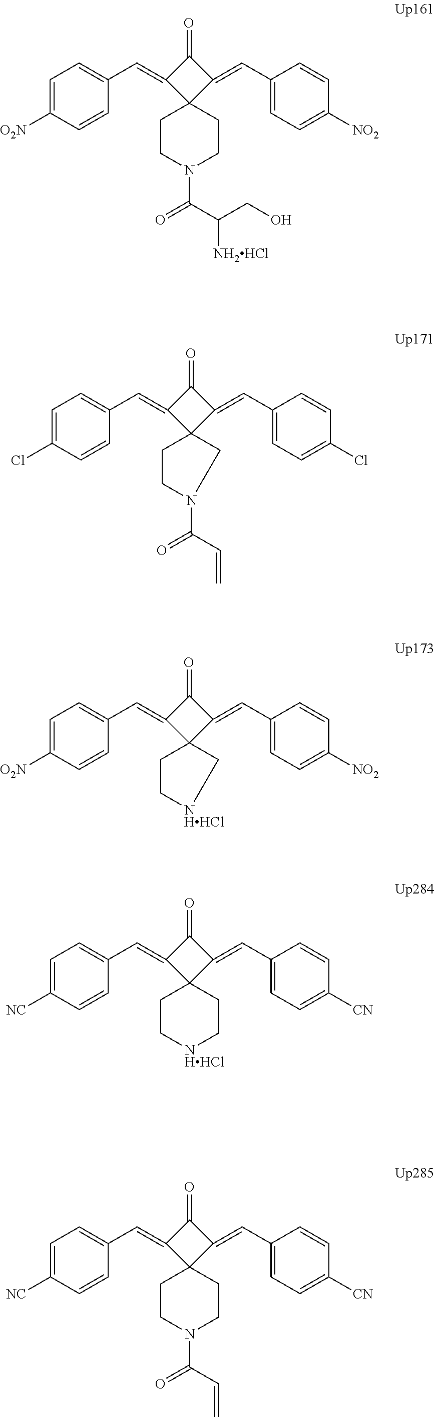

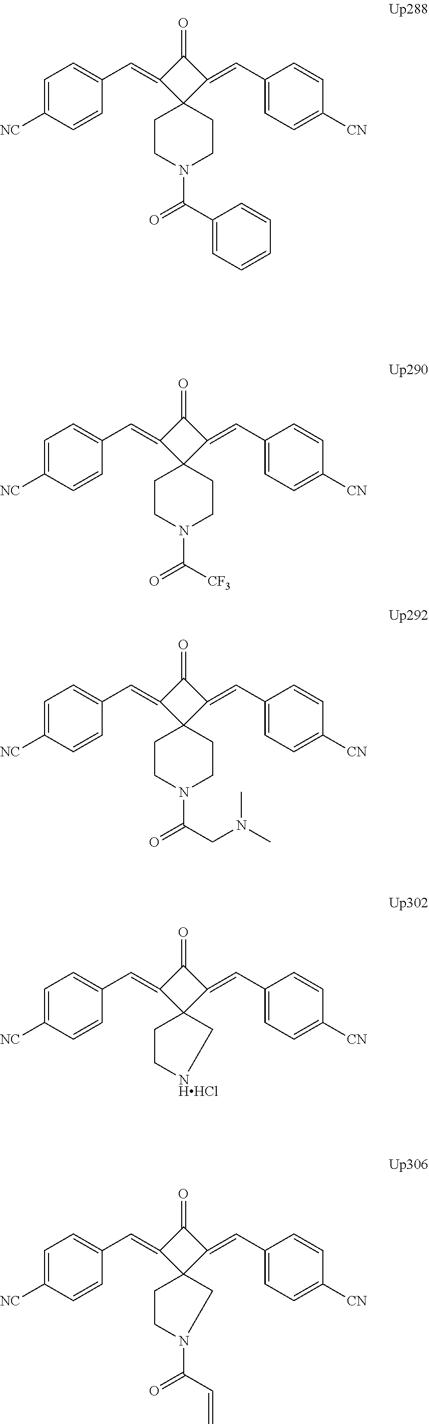

Also described herein are compounds represented by the structure of Formula I-III, where the compound is a free base or pharmaceutically acceptable salt, pharmaceutical composition, prodrug, isotope, geometrical isomer, solvate, metabolite, tautomer, N-oxide, polymorph, PROTAC or crystal.

Pharmaceutically acceptable salts are compounds that retain the biological effectiveness and properties of the free bases or free acids. In connection with the compounds described herein, salts of amines are formed with acids such as bromides, chlorides, bisulfates, borates, hydrobromates, hydrochlorates, sulfonates, sulfonic acids, thiocyanates, isothiocyanates and typically includes hydrochloric acid, hydrobromic acid (HBR), sulfuric acid, phosphoric acid, citric acid, mandelic acid, succinic acid and methanesulfonic acid, p-toluene sulfonic acid, benzene sulfonic acid, acetic acid, trifluoro acetic acid, and the like.

In other embodiment optically active salts such as (S)-mandelic acid, (R)- mandelic acid, (R)-(−)-α-methoxy-α-(trifluoromethyl)phenylacetic acid, (S)-(−)-α-methoxy-α-(trifluoromethyl)phenylacetic acid, (1S,3R)-(−)-camphoric acid, (1R,3S) -(+)-camphoric acid, L-(−) -malic acid, D-(+) -malic acid, or similarly well known in the art and can readily be adapted for use in connection with the compounds described herein.

Also described herein are pharmaceutical compositions including a pharmaceutically acceptable carrier and a compound or its salt form as described herein. Typically, the pharmaceutical composition described herein will comprise a compound described herein or its pharmaceutically acceptable salt, and a pharmaceutically acceptable carrier. Pharmaceutically acceptable carriers, as the term is used herein, refers to any suitable carriers, adjuvants, excipients, or stabilizers, and can be in solid or liquid form such as, tablets, capsules, powders, and/or solutions.

The compositions described herein can be used together with existing chemotherapy drugs to treat cancer. Typical chemotherapy drugs for use in connection with the compositions described herein include, for example, platinum agents (e.g., cisplatin, carboplatin or oxaliplatin), topo isomerase inhibitors (e.g., any form of doxorubicin), alkylating agents (e.g, Temozolomide), nitrosourea agents, plant derived alkaloids, hormone therapy treatments (e.g. androgen receptor inhibitors), aromatase inhibitors, P-glycoprotein inhibitors, other immunotherapeutic drugs, Lenalidomide and other anticancer agents.

In other embodiments the disease cancer can be any type of cancer and in particular ovarian cancer, carcinoma of the fallopian tubes, carcinoma of the endometrium, breast cancer, glioblastoma, brain cancers, brain stem glioma, carcinoma of the cervix, carcinoma of the vagina, carcinoma of the vulva, cancer of the head or neck, cancer of the esophagus, prostate cancer, colon cancer, lung cancer, non-small cell lung cancer (NSCLC), squamous cell carcinoma, bone cancer, pancreatic cancer, skin cancer, cutaneous or intraocular melanoma, uterine cancer, rectal cancer, cancer of the anal region, stomach cancer, Hodgkin's Disease, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, lymphocytic lymphomas, cancer of the bladder, cancer of the kidney or ureter, renal cell carcinoma, carcinoma of the renal pelvis, cancers of the central nervous system (CNS), neuroblastoma, primary CNS lymphoma, spinal axis tumors, pituitary adenoma, and combinations thereof.

The compounds according to Formulas I-Ill may be prepared using conventional organic synthetic methods. Exemplary synthetic routes are depicted below in the following general reaction schemes.

The skilled artisan will appreciate that if a substituent or solvent or related reagent described herein is not compatible with the synthetic methods described herein, the substituent or solvent or related reagent may be protected (in related to substituent) or replaced (in related to solvents and reagents) with a suitable protecting groups or solvents or reagents that are stable to the reaction conditions. The protecting groups may be removed at a suitable point in the reaction sequence to provide a desired intermediate or target compound.

The following abbreviations are used and have the indicated definitions: MHz is megahertz (frequency), m is multiplet, d is doublet, s is singlet, CDCl3 is deuterated chloroform, DMSO-d6 is deuterated DMSO, min is minutes, h is hours, g is grams, mg is milli grams, mmol is milli moles, mL is milliliters, μL is micro liters, N is normality, M is molarity, μM is micro molar, nM is nano molar, 0C is centigrade, TLC is thin layer chromatography, NMR is Nuclear Magnetic Resonance, ESI MS is Electro spray ionization mass spectrometry, DMSO is dimethyl sulfoxide, DCM is dichloromethane, DMF is N,N-dimethyl formamide, THF is tetrahydrofuran, NaHCO3 is sodium bicarbonate, RT is room temperature, EtOH is ethanol, NaOH is sodium hydroxide, KOH is potassium hydroxide, Et3N is triethyl amine, DIPEA is diisopropylethylamine, HCl is hydrogen chloride or hydrochloric acid, AcOH is acetic acid, TFA is trifluoro aceticacid, HOBT is N-hydroxy-benzotriazole, HBTU is 2-(1H-benzotriazol-1-yl)-1,1,3,3-tetramethyluronium hexafluorophosphate, Boc-Phe-OH is N-(tert-Butoxycarbonyl)-L-phenylalanine, Boc-L-phenylalanine,

### EXAMPLES

### Description of Synthesis

General Synthetic Procedure A:

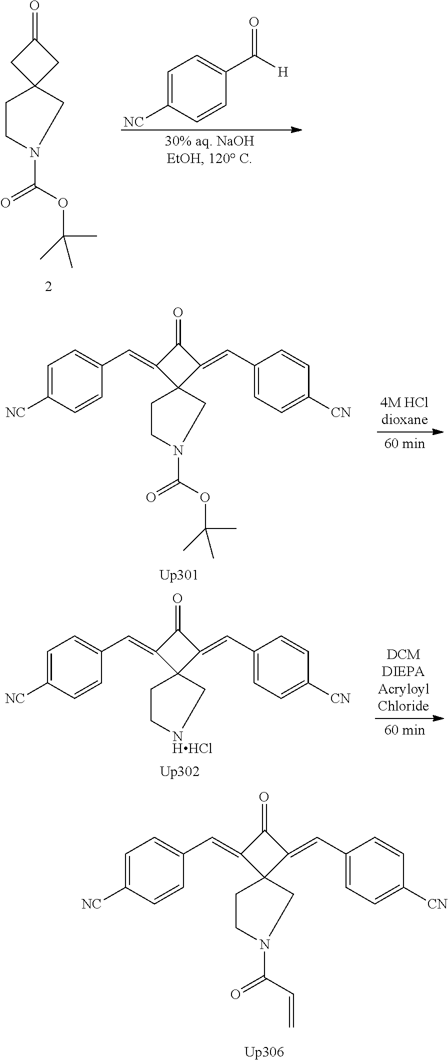

To a stirred solution of compound GA (1 eq) in any alcohol (example: Ethanol) were added a base such as NaOH (4 eq) or KOH (4 eq) dissolved in water and an aldehyde (RCHO) (2eq) and the reaction mixture was stirred at room temperature until TLC shows completion of the reaction. Water was added to the reaction mixture and the precipitated yellow solid was filtered, washed with water and dried under vacuum and purified by column chromatography over silica gel using ethyl acetate: hexanes mixture or crystallized in organic solvents to give the title compound GB which was dissolved in dioxane or any other solvents and treated with an acid (example 4M HCl in dioxane) and stirred at room temperature until the TLC show the completion of the reaction. Solvents were removed under vacuum and the solids were precipitated with diethyl ether or hexanes or any other solvents and dried under vacuum to give the title compound GC.

General Synthetic Procedure B:

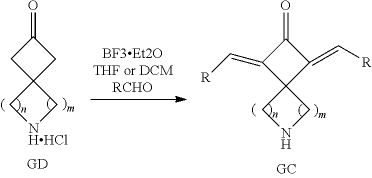

Compound GD (1 eq) in THF or DCM (dichloromethane) was treated with Boron trifluoride diethyl etherate (4-8 eq) dropwise at 00C, then aldehyde (2eq) was added to the reaction mixture in one portion and stirred overnight at room temperature. The reaction was quenched with a 10% aq. sodium bicarbonate solution and the yellow solid precipitate was filtered, washed with water and ethanol afforded compound GC. The typical yield in this procedure is 30-55%.

General synthetic procedure C:

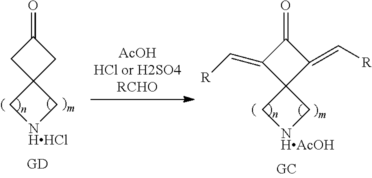

Compound GD (0.5 g, 1 eq) was dissolved in 10 mL acetic acid and added RCHO (2 eq). To this reaction mixture either dryHCL (generated from NaCl and H2SO4) gas bubbled for 15 min or direct H2SO4 (2 mL) was added in two portions and the reaction mixture stirred for overnight at room temperature. Solids were filtered off and washed with cold ethanol and dried under vacuum to afford compound GC. Typical yield in this procedure is 45-66%

Synthetic Example 1:Synthesis of Up284: To a stirred solution of 2-Oxo-7-azaspiro[3.5]nonane-7-carboxylate tert-butyl ester 1 (1.008, 1 eq) in 20 mL Ethanol were added aq.30% Sodium Hydroxide solution (4 eq, 2.4 mL) and 4-cyano benzaldehyde (1.18 g, 2 eq) at room temperature and stirring continued for 120 min. Top the reaction mixture water was added and the precipitated yellow solid was filtered, dried under vacuum and purified by column chromatography over silica gel using hexanes: ethyl acetate (8:2) as eluents. Removal of solvents under reduced pressure afforded Up283 as yellow solid (1.6 g, 82.4%). To a stirred solution of Up283 (1.2 g) in dioxane (10 mL) was added 4M HCl in dioxane (10 mL) and stirring continued for 60 min at room temperature. Solvents were removed under vacuum and the resultant yellow precipitate was precipitated with diethyl ether and dried under vacuum to afford Up284 (0.78 g, 75.7%) as yellow powder. 1HNMR (DMSO-d6, 400 MHz): δ9.09 (s, 1 H); 8.3 (d, 4 H); 7.93 (d, 4 H); 7.16 (s,2 H); 3.46 (s, 4 H); 2.2 (s, 4 H). ESI MS (m/z): 366 (M+1).

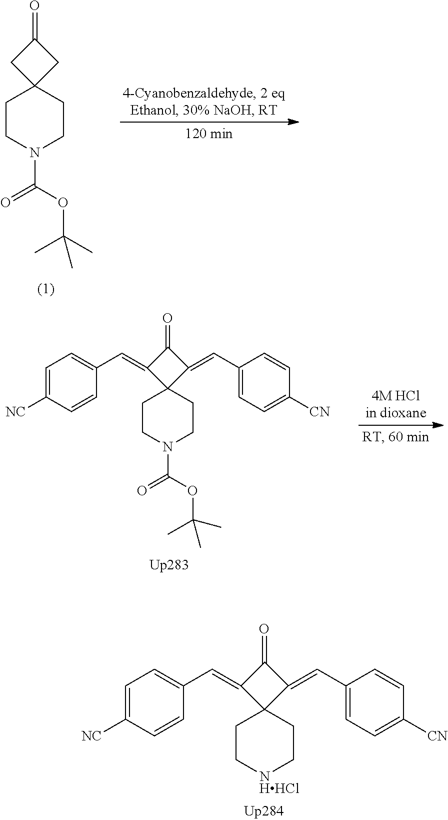

Synthetic Example 2: Synthesis of Up285: To a stirred solution of Up284 (0.2 g, 1 eq) in dichloromethane (10 mL) was added diisopropyl ethylamine (0.27 mL, 3 eq) followed by acryloyl chloride (43 μL, 1.05 eq) at 00C and stirred for 60 min at room temperature. Reaction completion was monitored by TLC. The reaction mixture was washed with saturated sodium bicarbonate aqueous solution (20 mL), extracted the compound with dichloromethane, dried over sodium sulfate, filtered and concentrated. The crude product was purified by column chromatography over silica gel using hexanes: ethyl acetate (4:6) as eluents to give the title compound Up285 (76%, 0.154 g) as an yellow powder. 1HNMR (CDCl3, 400 MHz): 8.1 (d, 4 H); 7.72 (d, 4 H); 6.67-6.72 (m, 3 H); 6.23-6.45 (m, 1 H); 5.71-5.83 (m, 1 H); 3.66-3.94 (m, 4H); 1.99-2.02 (m, 4 H). ESI MS (m/z): 366 (M+1).

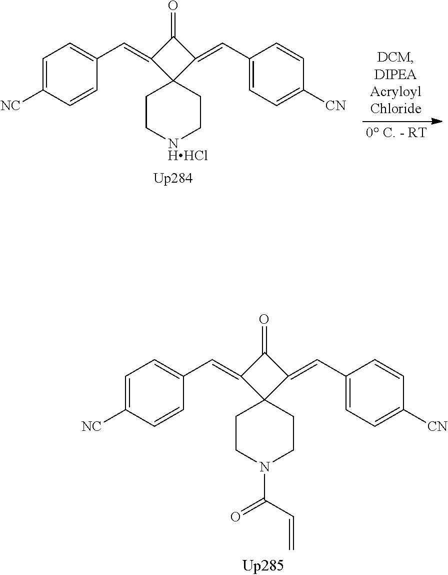

Synthetic Example 3: Synthesis of Up109: 2-Oxo-7-azaspiro[3.5]nonane-7-carboxylate tert-butyl ester 1 (1.2 g, 1 eq) in Ethanol (20 mL) was added 30% Sodium Hydroxide aq.solution (4 eq,) at room temperature and 4-nitro benzaldehyde (1.51g, 2 eq) was added and stirred the reaction mixture at room temperature for 120 min. Water was added and the yellow precipitate was filtered, dried under vacuum and purified by column chromatography using hexanes: ethyl acetate (8:2). Removal of solvents under reduced pressure afforded Up101 as yellow solid (2.04 g, 80.4%). 1HNMR (CDCl3, 400 MHz): 8.22-8.45 (m, 8 H), 6.72 (s, 2 H), 3.79-3.82 (m, 4 H), 2.02-2.12(m, 4 H), 1.67 (s, 9 H). Up101 (1.82 g) was dissolved in dioxane (10 mL) and treated with 4M HCl in dioxane (10 mL) and stirred at room temperature for 60 min. Solvents were removed under vacuum and the resultant yellow precipitate was washed with diethyl ether and dried under vacuum to afford Up104 as yellow powder (0.772 g, 77.2%) ESI MS (m/z) 405.9. Up104 (0.6 g, 1 eq) was dissolved in dichloromethane (15 mL) at 00C and and triethyl amine (0.59 mL, 3 eq) was added and stirred for 5 min. To this mixture acryloyl chloride (108 μL,1.05 eq) was added dropwise and stirred the reaction mixture for 60 min. Reaction completion was monitored by TLC and 8% sodium bicarbonate aqueous solution was added and the organic layer was separated and washed with water, brine and dried over sodium sulfate. Removal of solvents under vacuum afforded yellow precipitate which was purified by column chromatography using hexanes:ethyl acetate (4:6) afforded Up109 (0.494g, 78.5%) as an yellow powder. 1HNMR (CDCl3, 400 MHz): 8.14-8.48 (m, 8 H); 6.67-6.89 (m, 3 H); 6.33-6.41 (m, 1 H); 5.78-5.81 (m, 1 H); 3.79-4.09 (m, 4 H); 2.01-2.12 (m, 4 H). ESI MS (m/z): 459.2.

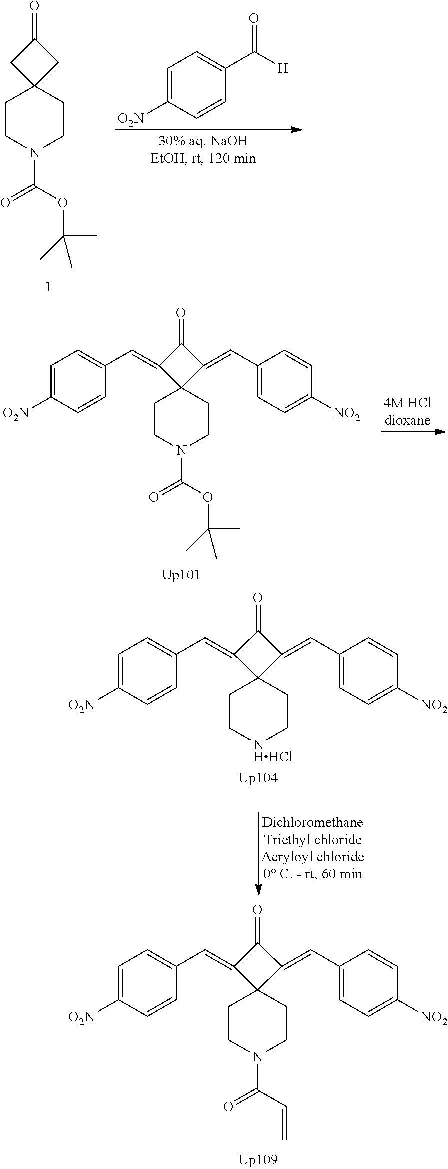

Synthetic Example 4: Synthesis of Up148: To a stirred solution of 2-Oxo-7-azaspiro[3.5]nonane-7-carboxylate tert-butyl ester 1 (0.21 g, 1 eq) in Ethanol (10 mL) were added 30% Sodium Hydroxide aq.solution (4 eq,) and 2-thiazolecarboxaldehyde (0.374 g, 2 eq) at room temperature and stirring continued for 120 min. Water was added to the reaction mixture and the precipitated yellow solid was filtered, dried under vacuum and purified by column chromatography over silica gel using hexanes: ethyl acetate (8:2) as eluents. Removal of solvents under reduced pressure afforded Up147 as yellow solid (0.28 g, 74.4%). To a stirred solutionjh of Up147 (0.15 g) in dioxane (3 mL) was added 4M HCl in dioxane (3 mL) and stirring continued for 60 min at room temperature. Solvents were removed under vacuum and the resultant yellow precipitate was washed with diethyl ether and dried under vacuum to afford Up148 as yellow powder (0.082 g, 65.3%) ESI MS (m/z) 330.

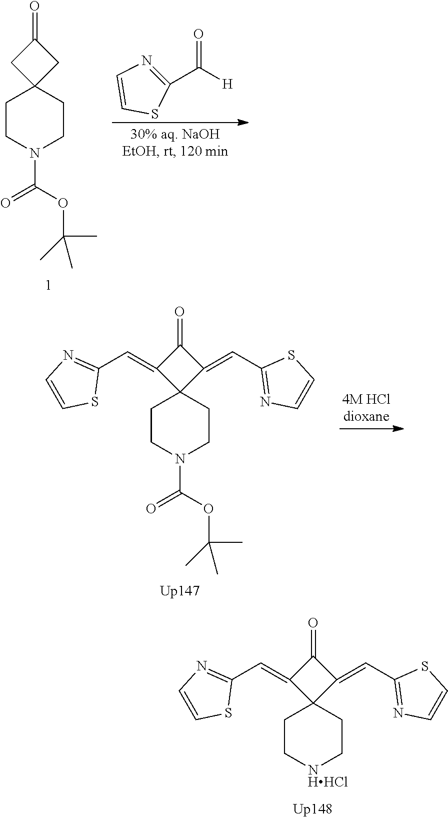

Synthetic Example 5: Synthesis of Up106: To a stirred solution of Boc-Phe-OH (0.172 g,1 eq) in DMF (5 mL) were added HBTU (0.246g, g, 1.1 eq), HOBt (0.085g, 1.1eq) and DIPEA (0.3 mL, 3 seq) sequentially and stirring continued for 5 min. Up104 (0.26 g, 1 eq, in DMF) was added to the reaction mixture and stirring continued for overnight. Water was added to the reaction mixture and extracted with ethyl acetate two times. Organic layers were combined, washed with 10% sodium bicarbonate solution, saturated ammonium chloride, water and brine and dried over sodium sulfate. Removal of solvents under reduced pressure afforded Up105 as a crude compound which was purified by column chromatography over silica gel using hexanes: Ethyl acetate mixture (6:4) as eluents to give the title compound Up105 as a yellow solid (0.31 g 79.8%). To a stirred solution of Up105 (0.24 g) in dioxane (4 mL) was added 4M HCl in dioxane (4 mL) and stirring continued for 60 min. Solvents were removed under vacuum and the crude compound was precipitated in diethylether and dried under vacuum to give the title compound Up106 (0.17 g, 80.5%). ESIMS (m/z): 553.

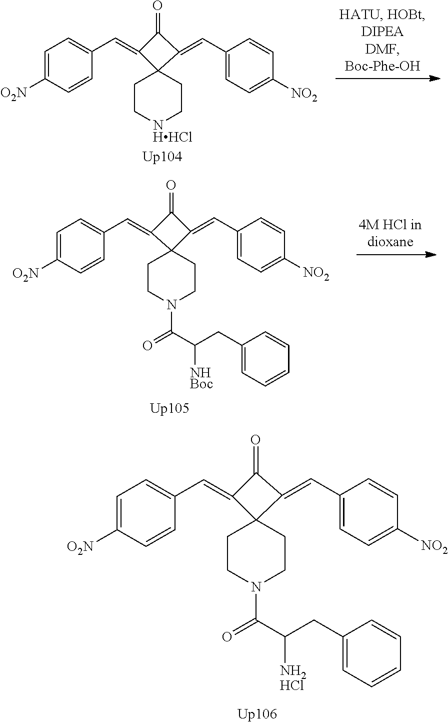

Synthetic Example 6: Synthesis of Up108 & Up112: To a stirred solution of 2-Oxo-7-azaspiro[3.5]nonane-7-carboxylate tert-butyl ester 1 (0.41 g, 1 eq) in Ethanol (10 mL) were added 30% Sodium Hydroxide aq.solution (4 eq,) and 3,4-dichlorobenzaldehyde (0.59 g, 2 eq) at room temperature and stirring continued for 120 min. Water was added to the reaction mixture and the precipitated yellow solid was filtered, dried under vacuum and purified by column chromatography over silica gel using hexanes: ethyl acetate (8:2) as eluents. Removal of solvents under reduced pressure afforded Up107 as yellow solid (0.77 g, 81.1%). 1HNMR (CDCl3, 400 MHz):δ8.2 (s, 2 H), 7.92-7.99 (d, 2 H), 7.52-7.6(d, 2 H), 6.51 (d, 2 H), 3.69-3.72(m, 4 H), 1.97-2.01(m, 4 H), 1.69 (s, 9 H).To a stirred solution of Up107 (0.55 g) in dioxane (8 mL) was added 4M HCl in dioxane (8 mL) and stirring continued for 60 min at room temperature. Solvents were removed under vacuum and the resultant yellow precipitate was washed with diethyl ether and dried under vacuum to afford Up108 as yellow powder (0.41 g, 84.3%). 1HNMR (CD3OD, 400 MHz):δ8.7 (s, 2 H), 8.18-8.26 (d, 2 H), 7.71-7.79 (d, 2 H), 6.9 (s, 2 H), 3.56-3.69 (m, 4 H), 2.21-2.25 (m, 4H); ESI MS (m/z) 454 (M+1). To a stirred solution of Up108 (0.21 g, 1 eq) in dichloromethane (10 mL) was added di isopropyl ethylamine (0.22 mL, 3 eq) followed by acryloyl chloride (37 μL, 1.05 eq) at 00C and stirred for 60 min at room temperature. Reaction completion was monitored by TLC. The reaction mixture was washed with saturated sodium bicarbonate aqueous solution (20 mL), extracted the compound with dichloromethane, dried over sodium sulfate, filtered and concentrated. The crude product was purified by column chromatography over silica gel using hexanes: ethyl acetate (4:6) as eluents to give the title compound Up112 (76%, 0.154 g) as an yellow powder (0.17 g, 78.3%). 1HNMR (CDCl3, 400 MHz):δ8.15 (s, 2 H), 7.89-7.92 (d, 2 H), 7.41-7.55 m(d, 2 H), 6.55-6.61 (m, 1 H), 6.46 (s, 2 H), 6.27-6.31 (d, 1 H), 5.68-5.72 (d, 1 H), 3.71-3.9 (m, 4 H), 1.88-1.98(m, 4 H).

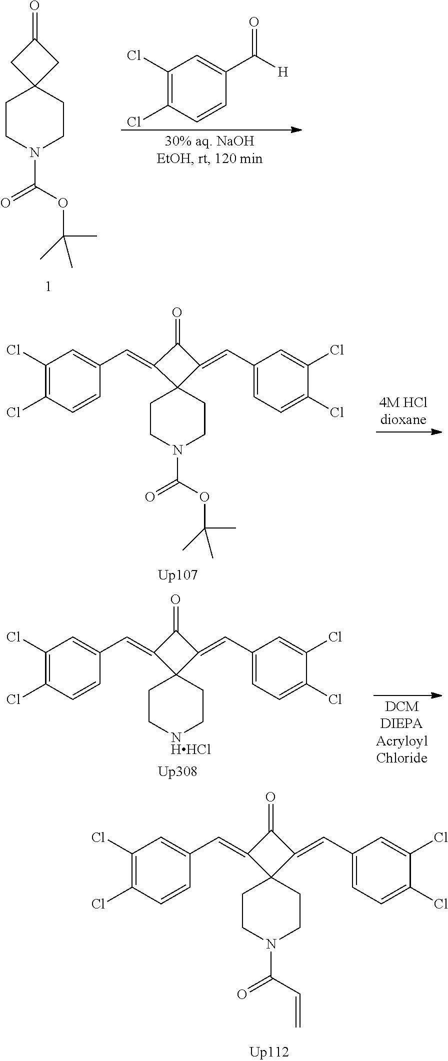

Synthetic Example 7: Synthesis of Up161:

To a stirred solution of Boc-O-tert-butyl-L-serine (0.124 g, 1 eq) in DMF (5 mL) were added HBTU (0.18 g, 1.1 eq), HOBt (0.062 g, 1.1 eq) and DIPEA (230 μL, 3 eq) sequentially and stirring continued for 5 min. Up104 (0.19 g, 1 eq, in DMF) was added to the reaction mixture and stirring continued for overnight. Water was added to the reaction mixture and extracted with ethyl acetate two times. Organic layers were combined, washed with 10% sodium bicarbonate solution, saturated ammonium chloride, water and brine and dried over sodium sulfate. Removal of solvents under reduced pressure afforded Up160 as a crude compound which was purified by column chromatography over silica gel using hexanes: Ethyl acetate mixture (7:3) as eluents to give the title compound Up160 as a yellow solid (0.19 g, 68.1%). To a stirred solution of Up160 (0.13 g) in dioxane (4 mL) was added 4M HCl in dioxane (8 mL) and stirring continued for 48 hrs at room temperature. Solvents were removed under vacuum and the resultant yellow precipitate was washed with diethyl ether and dried under vacuum to afford Up161 as yellow powder (0.026 g, 36.3%). ESIMS (m/z): 588 (M+CH3CN:H2O)

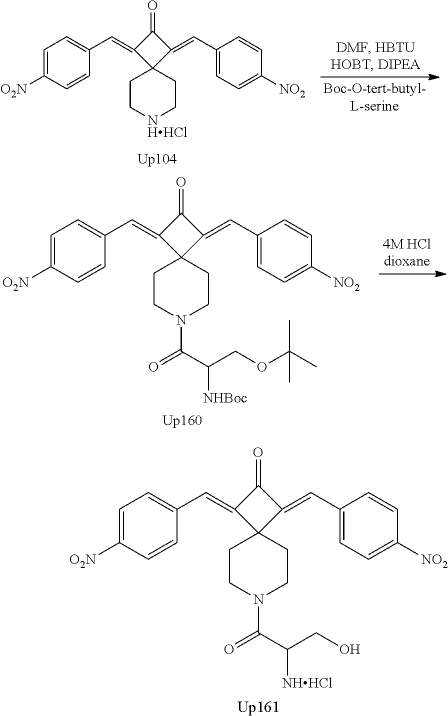

Synthetic Example 8: Synthesis of Up200: To a stirred solution of Probenecid (0.142 g, 1 eq) in DMF (3 mL) were added HBTU (0.2 g, 1.1 eq), HOBt (0.074 g, 1.1 eq) and DIPEA (253 μL, 3 eq) sequentially and stirring continued for 5 min. Up104 (0.22 g, 1 eq, in DMF) was added to the reaction mixture and stirring continued for overnight. Water was added to the reaction mixture and extracted with ethyl acetate two times. Organic layers were combined, washed with 10% sodium bicarbonate solution, saturated ammonium chloride, water and brine and dried over sodium sulfate. Removal of solvents under reduced pressure afforded Up200 as a crude compound which was purified by column chromatography over silica gel using hexanes: Ethyl acetate mixture (7:3) as eluents to give the title compound Up200 as a yellow solid (0.22 g, 65.6%). ESIMS (m/z): 690.9 (M+NH3).

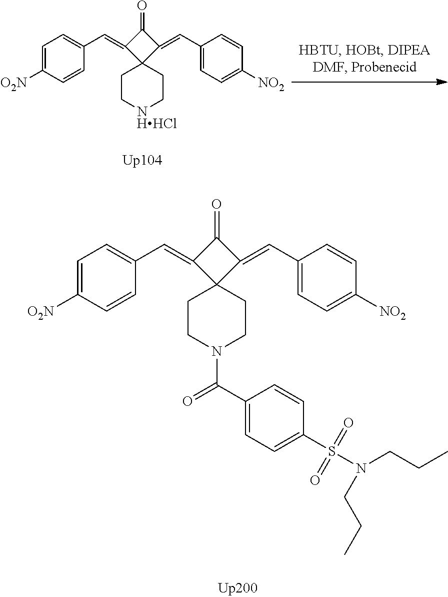

Synthetic Example 9: Synthesis of Up173: To a stirred solution of 2-Oxo-6-azaspiro[3.4]octane-6-carboxylate tert-butyl ester 2 (0.27 g, 1 eq) in 20 mL Ethanol were added aq.30% Sodium Hydroxide solution (4 eq,) and 4-nitro benzaldehyde (0.362 g, 2 eq) at room temperature and stirring continued for 120 min. To the reaction mixture water was added and the precipitated yellow solid was filtered, dried under vacuum and purified by column chromatography over silica gel using hexanes: ethyl acetate (8:2) as eluents. Removal of solvents under reduced pressure afforded Up172 as yellow solid (0.43 g, 73.3%). 1HNMR (CDCl3, 400 MHz): δ8.23-8.41 (m, 8 H); 6.62 (s, 2 H); 3.66-3.79 (m, 4 H); 2.3-2.39 (m, 2 H), 1.52 (s, 9 H) To a stirred solution of Up172 (0.36 g, 1 eq) in dioxane (5 mL) was added 4M HCl in dioxane (5 mL) and stirring continued for 60 min at room temperature. Solvents were removed under vacuum and the resultant yellow precipitate was precipitated with diethyl ether and dried under vacuum to afford Up173 (0.28 g, 75.06%) as yellow powder. ESI MS (m/z): 392 (M+1)

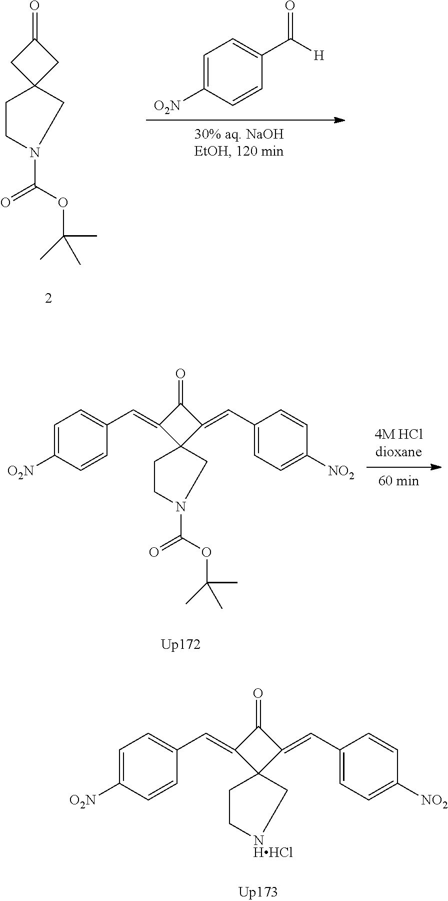

Synthetic Example 10: Synthesis of Up171: To a stirred solution of 2-Oxo-6-azaspiro[3.4]octane-6-carboxylate tert-butyl ester 2 (0.16 g, 1 eq) in 5 mL Ethanol were added aq.30% Sodium Hydroxide solution (4 eq,) and 4-chloro benzaldehyde (0.199 g, 2 eq) at room temperature and stirring continued for 120 min. To the reaction mixture water was added and the precipitated light yellow solid was filtered, dried under vacuum and purified by column chromatography over silica gel using hexanes: ethyl acetate (8:2) as eluents. Removal of solvents under reduced pressure afforded Up168 as yellow solid (0.261 g, 78.1%). To a stirred solution of Up168 (0.22 g) in dioxane (4 mL) was added 4M HCl in dioxane (4 mL) and stirring continued for 60 min at room temperature. Solvents were removed under vacuum and the resultant yellow precipitate was precipitated with diethyl ether and dried under vacuum to afford Up169 (0.116 g, 61%) as yellow powder. ESI MS (m/z): 370 (M+1). To a stirred solution of Up169 (81 mg, 1 eq) in DCM (5 mL) were added DIPEA (98 μL, 3 eq) and Acryloyl chloride (17 μL, 1 eq) at 00C and stirring continued for 60 min at room temperature. Reaction completion was monitored by TLC. The reaction mixture was washed with saturated sodium bicarbonate aqueous solution (20 mL), extracted the compound with dichloromethane, dried over sodium sulfate, filtered and concentrated. The crude product was purified by column chromatography over silica gel using hexanes: ethyl acetate (4:6) as eluents to give the title compound Up171 (66.1%, 56.1 mg) as an yellow powder. ESI MS (m/z): 424 (M+1).

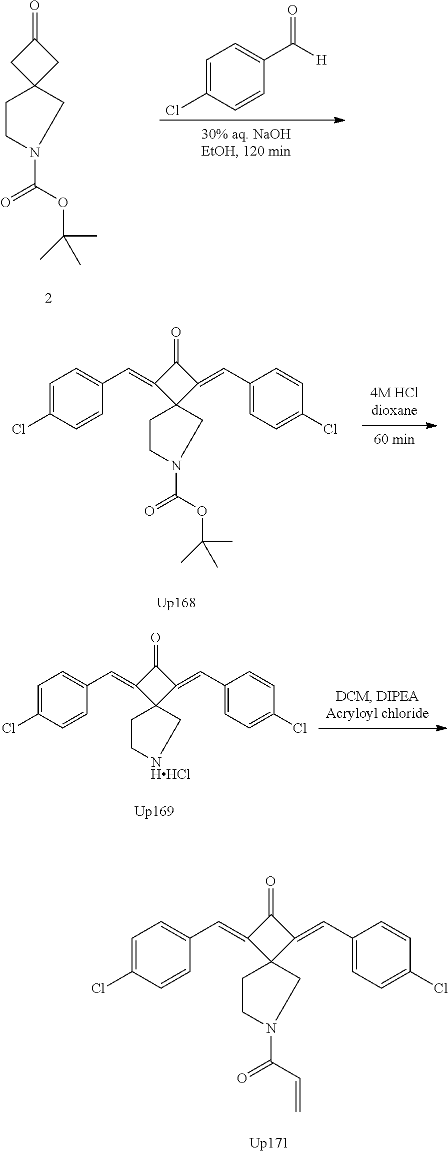

Synthetic Example 11: Synthesis of Up302 and Up306: To a stirred solution of 2-Oxo-6-azaspiro[3.4]octane-6-carboxylate tert-butyl ester 2 (1.008, 1 eq) in 20 mL Ethanol were added aq.30% Sodium Hydroxide solution (4 eq, 2.4 mL) and 4-cyano benzaldehyde (1.18 g, 2 eq) at room temperature and stirring continued for 120 min. To the reaction mixture water was added and the precipitated light yellow solid was filtered, dried under vacuum and purified by column chromatography over silica gel using hexanes: ethyl acetate (8:2) as eluents. Removal of solvents under reduced pressure afforded Up301 as yellow solid (1.6 g, 82.4%). To a stirred solution of Up301 (1.2 g) in dioxane (10 mL) was added 4M HCl in dioxane (10 mL) and stirring continued for 60 min at room temperature. Solvents were removed under vacuum and the resultant yellow precipitate was precipitated with diethyl ether and dried under vacuum to afford Up302 (0.78 g, 75.7%) as yellow powder. ESI MS (m/z): 352 (M+1). To a stirred solution of Up302 (0.21 g, 1 eq) in dichloromethane (10 mL) was added di isopropyl ethylamine (0.22 mL, 3 eq) followed by acryloyl chloride (37 μL, 1.05 eq) at OOC and stirred for 60 min at room temperature. Reaction completion was monitored by TLC. The reaction mixture was washed with saturated sodium bicarbonate aqueous solution (20 mL), extracted the compound with dichloromethane, dried over sodium sulfate, filtered and concentrated. The crude product was purified by column chromatography over silica gel using hexanes: ethyl acetate (4:6) as eluents to give the title compound Up306 (76%, 0.154 g) as an yellow powder (0.17 g, 78.3%).

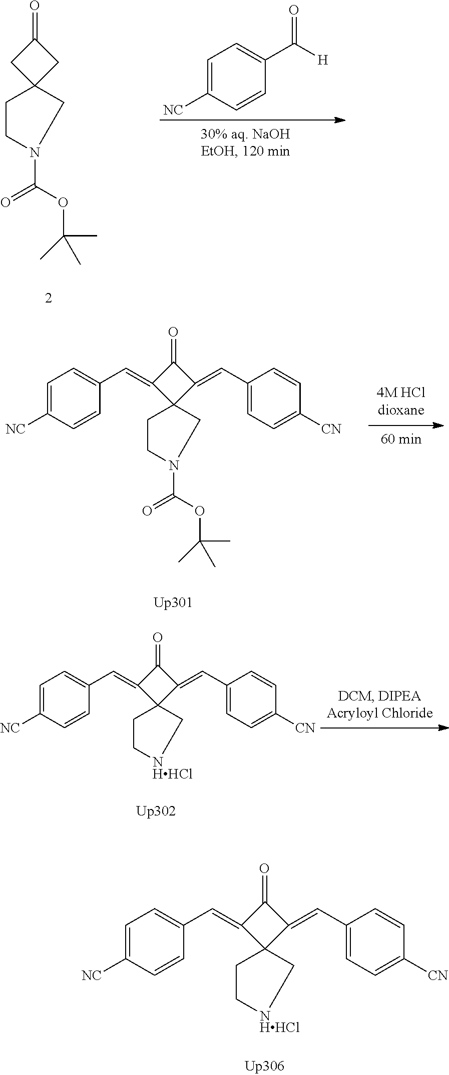

Synthetic Example 12: Synthesis of Up188: To a stirred solution of Up104 (76 mg, 1 eq) in DCM (5 mL) were added DIPEA (90 μL, 3 eq) and benzenesulfonyll chloride (22 μL, 1 eq) at 00C and stirring continued for 60 min at room temperature. Reaction completion was monitored by TLC. The reaction mixture was washed with saturated sodium bicarbonate aqueous solution (10 mL), extracted the compound with dichloromethane, dried over sodium sulfate, filtered and concentrated. The crude product was purified by column chromatography over silica gel using hexanes:ethyl acetate (4:6) as eluents to give the title compound Up188 (71.2%, 67 mg) as an yellow powder. ESI MS (m/z): 546 (M+1)

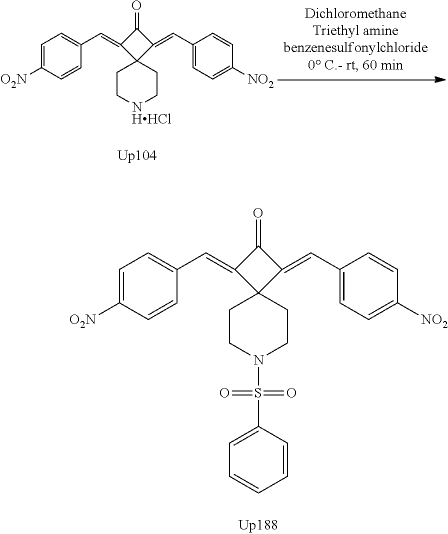

Synthetic Example 13: Synthesis of Up288: To a stirred solution of Up284 (56.6 mg,1 eq) in DCM (5 mL) were added DIPEA (75 μL, 3 eq) and benzoyl chloride (22.2 μL,1 eq) at 00C and stirring continued for 60 min at room temperature. Reaction completion was monitored by TLC. The reaction mixture was washed with saturated sodium bicarbonate aqueous solution (10 mL), extracted the compound with dichloromethane, dried over sodium sulfate, filtered and concentrated. The crude product was purified by column chromatography over silica gel using hexanes:ethyl acetate (1:1) as eluents to give the title compound Up288 (61.5%, 42 mg) as an yellow powder. ESI MS (m/z): 470 (M+1)

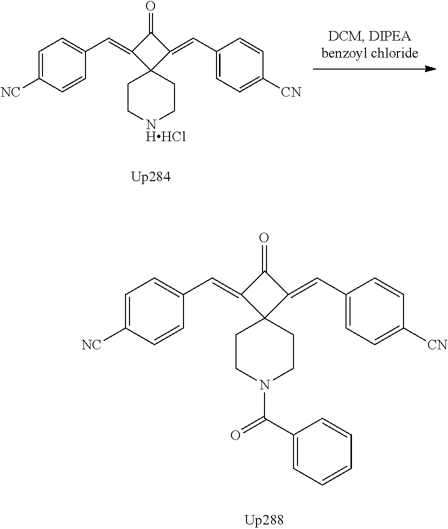

Synthetic Example 14: Synthesis of Up290: To a stirred solution of Up284 (53.2 mg, 1 eq) in DCM (5 mL) were added DIPEA (724, 3 eq) and Trifluoroacetic anhydride (19 μL,1 eq) at 00C and stirring continued for 60 min at room temperature. Reaction completion was monitored by TLC. The reaction mixture was washed with saturated sodium bicarbonate aqueous solution (20 mL), extracted the compound with dichloromethane, dried over sodium sulfate, filtered and concentrated. The crude product was purified by column chromatography over silica gel using hexanes:ethyl acetate (7:3) as eluents to give the title compound Up290 (74.6%, 47 mg) as an yellow powder. ESI MS (m/z): 462 (M+1).

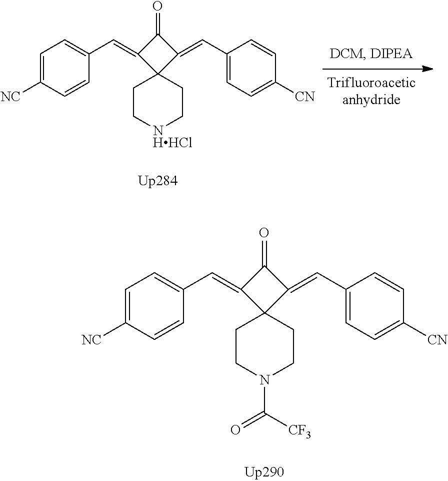

Synthetic Example 15: Synthesis of Up199 and Up201: To a stirred solution of 2-Oxo-7-azaspiro[3.5]nonane-7-carboxylate tert-butyl ester 1 (0.24 g, 1 eq) in Ethanol (10 mL) were added 30% Sodium Hydroxide aq.solution (4 eq,) and 4-bromobenzaldehyde (0.371 g, 2 eq) at room temperature and stirring continued for 120 min. Water was added to the reaction mixture and the precipitated yellow solid was filtered, dried under vacuum and purified by column chromatography over silica gel using hexanes: ethyl acetate (8:2) as eluents. Removal of solvents under reduced pressure afforded Up197 as yellow solid (0.484 g, 85.3%). To a stirred solution of Up197 (0.4 g) in dioxane (6 mL) was added 4M HCl in dioxane (6 mL) and stirring continued for 60 min at room temperature. Solvents were removed under vacuum and the resultant yellow precipitate was washed with diethyl ether and dried under vacuum to afford Up198 as yellow powder (0.29 g, 81.9%) ESI MS (m/z) 472. To a stirred solution of Up198 (0.11 g, 1 eq) in dichloromethane (6 mL) was added diisopropyl ethylamine (0.12 mL, 3 eq) followed by acryloyl chloride (19 μL, 1.05 eq) at 00C and stirred for 60 min at room temperature. Reaction completion was monitored by TLC. The reaction mixture was washed with saturated sodium bicarbonate aqueous solution (20mL), extracted the compound with dichloromethane, dried over sodium sulfate, filtered and concentrated. The crude product was purified by column chromatography over silica gel using hexanes: ethyl acetate (4:6) as eluents to give the title compound Up199 as an yellow powder (0.088 g, 77.1%) ESIMS (m/z: 528-M+1). To a stirred solution of Up198 in DMF (mL) were added K2CO3 and 1-bromomethylnaphthalene at room temperature and stirring continued for overnight at 900 C. Water was added to the reaction mixture and extracted with ethyl acetate, washed with brine, dried over sodium sulfate and concentrated under vacuum. The crude compound was purified by column chromatography over silica gel using hexanes:ethylacetate (8:2) to give the title compound Up201 as yellow solid. ESI MS (m/z: 631-M+H20).

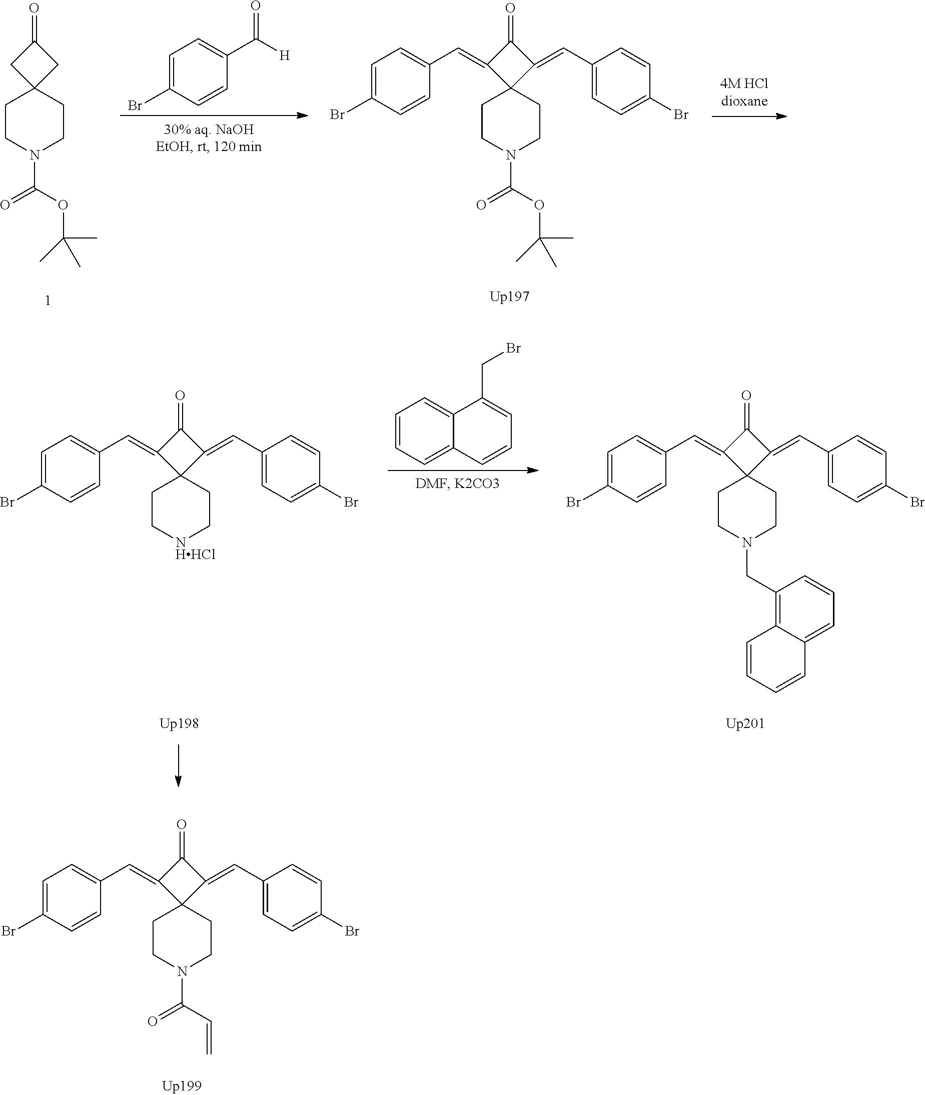

Synthetic Example 16: Synthesis of Up142: To a stirred solution of 2-Oxo-7-azaspiro[3.5]nonane-7-carboxylate tert-butyl ester 1 (0.17 g, 1 eq) in Ethanol (10 mL) were added 30% Sodium Hydroxide aq.solution (4 eq,) and benzaldehyde (0.263 g, 2 eq) at room temperature and stirring continued for 120 min. Water was added to the reaction mixture and the precipitated yellow solid was filtered, dried under vacuum and purified by column chromatography over silica gel using hexanes: ethyl acetate (8:2) as eluents. Removal of solvents under reduced pressure afforded Up138 as yellow solid (0.36 g, 78.9%). To a stirred solution of Up138 (0.3 g) in dioxane (6 mL) was added 4M HCl in dioxane (6 mL) and stirring continued for 60 min at room temperature. Solvents were removed under vacuum and the resultant yellow precipitate was washed with diethyl ether and dried under vacuum to afford Up140as yellow powder (0.18 g,71.1%) ESI MS (m/z) 316. To a stirred solution of Up140 (0.11 g, 1 eq) in dichloromethane (6 mL) was added diisopropyl ethylamine (0.16 mL, 3 eq) followed by acryloyl chloride (24 μL, 1.05 eq) at 00C and stirred for 60 min at room temperature. Reaction completion was monitored by TLC. The reaction mixture was washed with saturated sodium bicarbonate aqueous solution (20 mL), extracted the compound with dichloromethane, dried over sodium sulfate, filtered and concentrated. The crude product was purified by column chromatography over silica gel using hexanes: ethyl acetate (4:6) as eluents to give the title compound Up142 as an yellow powder (0.08 g, 68.9%) ESIMS (m/z: 370-M+1).

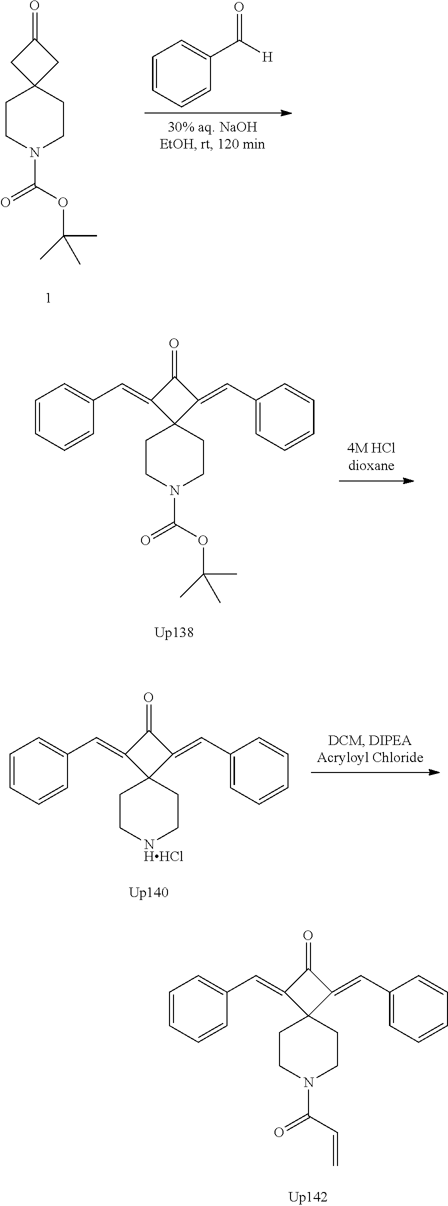

Synthetic Example 17: Synthesis of Up132: To a stirred solution of 2-Oxo-7-azaspiro[3.5]nonane-7-carboxylate tert-butyl ester 1 (0.2 g, 1 eq) in Ethanol (10 mL) were added 30% Sodium Hydroxide aq.solution (4 eq,) and 4-fluoro benzaldehyde (0.207 g, 2 eq) at room temperature and stirring continued for 120 min. Water was added to the reaction mixture and the precipitated yellow solid was filtered, dried under vacuum and purified by column chromatography over silica gel using hexanes: ethyl acetate (8:2) as eluents. Removal of solvents under reduced pressure afforded Up130 as yellow solid (0.26 g, 76.9%). To a stirred solution of Up130 (0.22 g) in dioxane (6 mL) was added 4M HCl in dioxane (6 mL) and stirring continued for 60 min at room temperature. Solvents were removed under vacuum and the resultant yellow precipitate was washed with diethyl ether and dried under vacuum to afford Up131 as yellow powder (0.137 g, 72.8%) ESI MS (m/z) 353. To a stirred solution of Up131 (0.086 g, 1 eq) in dichloromethane (4 mL) was added diisopropyl ethylamine (0.12 mL, 3 eq) followed by acryloyl chloride (18 μL, 1.05 eq) at OOC and stirred for 60 min at room temperature. Reaction completion was monitored by TLC. The reaction mixture was washed with saturated sodium bicarbonate aqueous solution (12 mL), extracted the compound with dichloromethane, dried over sodium sulfate, filtered and concentrated. The crude product was purified by column chromatography over silica gel using hexanes: ethyl acetate (4:6) as eluents to give the title compound Up132 as an yellow powder (0.062 g, 68.8%) ESIMS (m/z: 406-M+1).

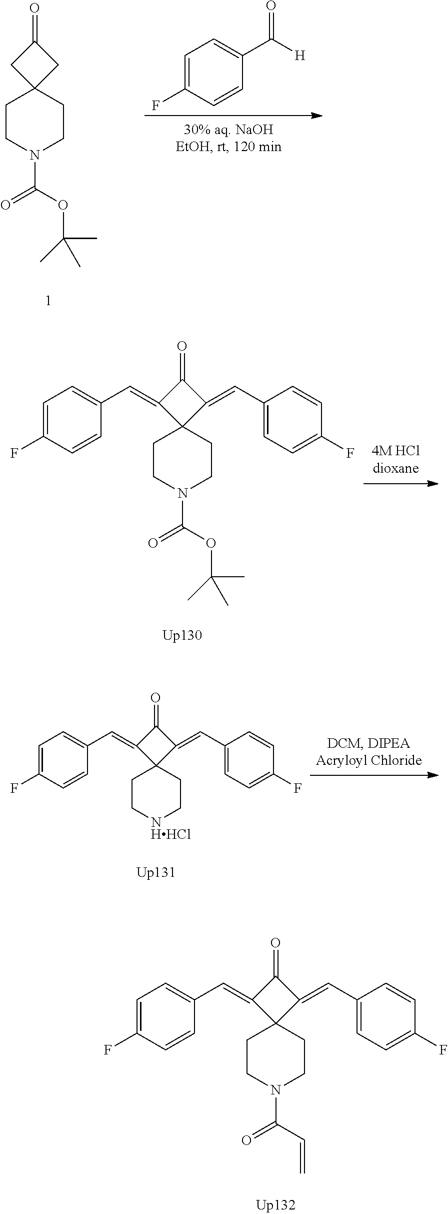

Synthetic Example 18: Synthesis of Up135: To a stirred solution of 2-Oxo-7-azaspiro[3.5]nonane-7-carboxylate tert-butyl ester 1 (0.24 g, 1 eq) in Ethanol (10 mL) were added 30% Sodium Hydroxide aq.solution (4 eq,) and 2-fluoro benzaldehyde (0.249 g, 2 eq) at room temperature and stirring continued for 120 min. Water was added to the reaction mixture and the precipitated yellow solid was filtered, dried under vacuum and purified by column chromatography over silica gel using hexanes: ethyl acetate (8:2) as eluents. Removal of solvents under reduced pressure afforded Up133 as yellow solid (0.347 g, 83.4%). To a stirred solution of Up133 (0.29 g) in dioxane (6 mL) was added 4M HCl in dioxane (6 mL) and stirring continued for 60 min at room temperature. Solvents were removed under vacuum and the resultant yellow precipitate was washed with diethyl ether and dried under vacuum to afford Up134 as yellow powder (0.183 g, 75%) ESI MS (m/z) 353. To a stirred solution of Up134 (0.124 g, 1 eq) in dichloromethane (4 mL) was added diisopropyl ethylamine (0.16 mL, 3 eq) followed by acryloyl chloride (25 μL, 1.05 eq) at OOC and stirred for 60 min at room temperature. Reaction completion was monitored by TLC. The reaction mixture was washed with saturated sodium bicarbonate aqueous solution (12 mL), extracted the compound with dichloromethane, dried over sodium sulfate, filtered and concentrated. The crude product was purified by column chromatography over silica gel using hexanes: ethyl acetate (4:6) as eluents to give the title compound Up135 as an yellow powder (0.087 g, 67.4%) ESIMS (m/z: 406-M+1).

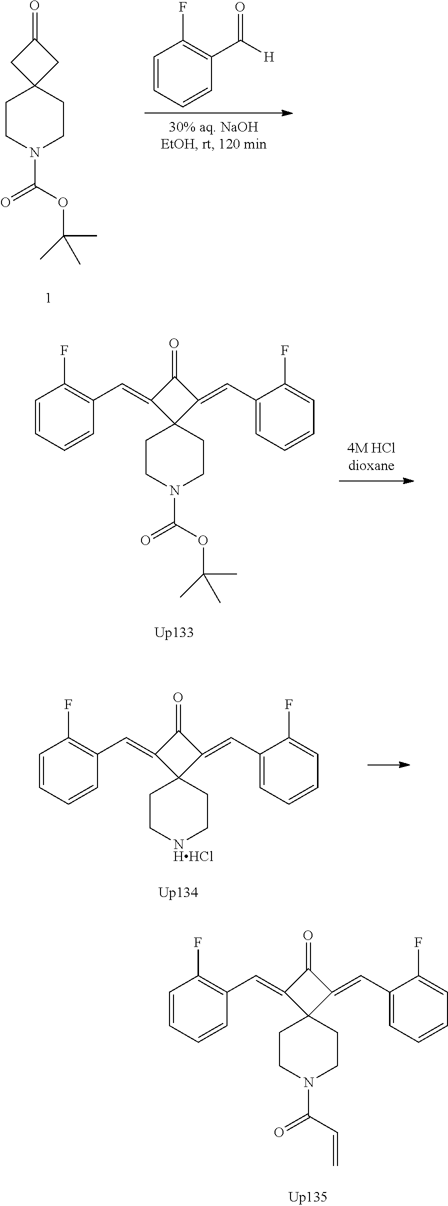

Synthetic Example 19: Synthesis of Up137: To a stirred solution of 2-Oxo-7-azaspiro[3.5]nonane-7-carboxylate tert-butyl ester 1 (0.12 g, 1 eq) in Ethanol (10 mL) were added 30% Sodium Hydroxide aq.solution (4 eq,) and cinnamaldehyde (0.132 g, 2 eq) at room temperature and stirring continued for 120 min. Water was added to the reaction mixture and the precipitated yellow solid was filtered, dried under vacuum and purified by column chromatography over silica gel using hexanes: ethyl acetate (8:2) as eluents. Removal of solvents under reduced pressure afforded Up136 as yellow solid (0.17 g, 72.64%). To a stirred solution of Up136 (0.15 g) in dioxane (4 mL) was added 4M HCl in dioxane (4 mL) and stirring continued for 60 min at room temperature. Solvents were removed under vacuum and the resultant yellow precipitate was washed with diethyl ether and dried under vacuum to afford Up137 as yellow powder (0.09 g, 69.7%) ESI MS (m/z) 368.

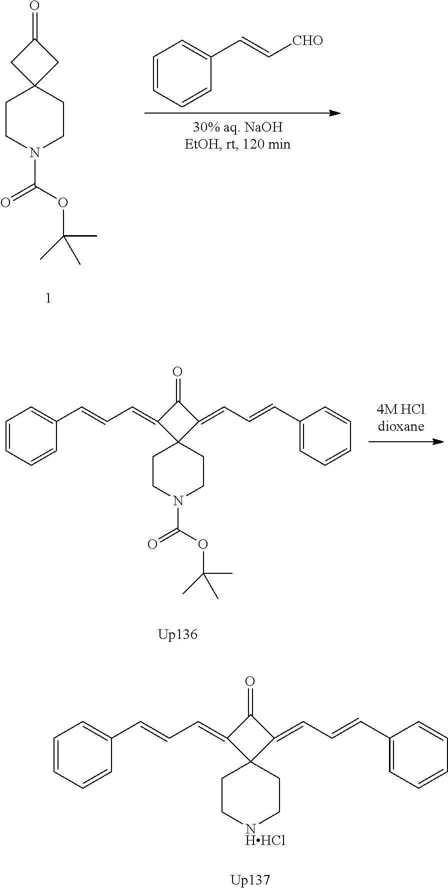

Synthetic Example 20: Synthesis of Up144: To a stirred solution of 2-Oxo-7-azaspiro[3.5]nonane-7-carboxylate tert-butyl ester 1 (0.18 g, 1 eq) in Ethanol (10 mL) were added 30% Sodium Hydroxide aq.solution (4 eq,) and picolinaldehyde (0.161 g, 2 eq) at room temperature and stirring continued for 120 min. Water was added to the reaction mixture and the precipitated yellow solid was filtered, dried under vacuum and purified by column chromatography over silica gel using hexanes: ethyl acetate (7:3) as eluents. Removal of solvents under reduced pressure afforded Up143 as yellow solid (0.23 g, 73.2%). To a stirred solution of Up143 (0.2 g) in dioxane (4 mL) was added 6M HCl in dioxane (6 mL) and stirring continued for 60 min at room temperature. Solvents were removed under vacuum and the resultant yellow precipitate was washed with diethyl ether and dried under vacuum to afford Up144 as yellow powder (0.18 g, 73.6%) ESI MS (m/z) 318.

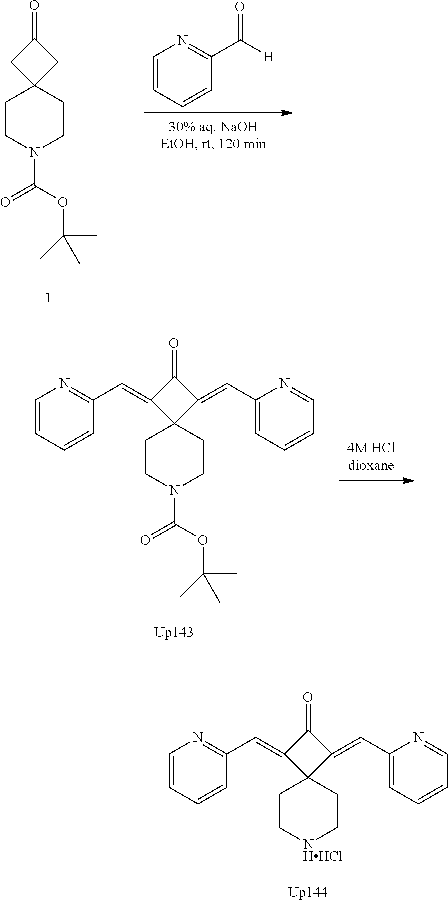

Synthetic Example 21: Synthesis of Up291: To a stirred solution of valproic acid (0.056 g, 1.1 eq) in DMF (5 mL) were added HBTU (0.146 g, 1.1 eq), HOBt (0.05 g, 1.1eq) and DIPEA (180 4, 3 eq) sequentially and stirring continued for 5 min. Up284 (0.14 g, 1eq, in DMF) was added to the reaction mixture and stirring continued for overnight. Water was added to the reaction mixture and extracted with ethyl acetate two times. Organic layers were combined, washed with 10% sodium bicarbonate solution, saturated ammonium chloride, water and brine and dried over sodium sulfate. Removal of solvents under reduced pressure afforded Up291 as a crude compound which was purified by column chromatography over silica gel using hexanes: Ethyl acetate mixture (6:4) as eluents to give the title compound Up291 as a yellow solid (0.13 g, 76%). ESIMS (m/z): 492 (M+1).

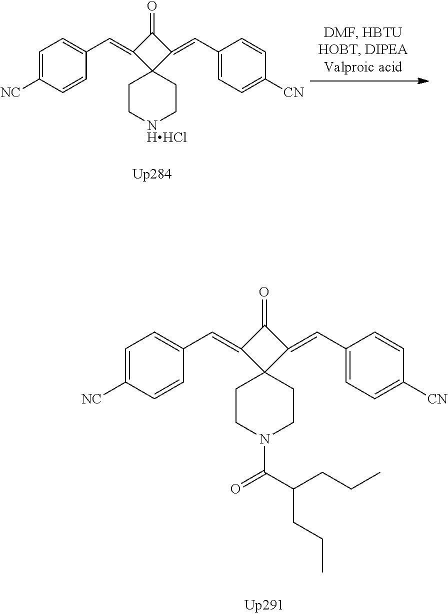

Synthetic Example 22: To a stirred solution of dimethylglycine (0.04 g, 1.1 eq) in DMF (5 mL) were added HBTU (0.146 g, 1.1 eq), HOBt (0.05 g, 1.1 eq) and DIPEA (180 μL, 3 eq) sequentially and stirring continued for 5 min. Up284 (0.14 g, 1eq, in DMF) was added to the reaction mixture and stirring continued for overnight. Water was added to the reaction mixture and extracted with ethyl acetate two times. Organic layers were combined, washed with 10% sodium bicarbonate solution, saturated ammonium chloride, water and brine and dried over sodium sulfate. Removal of solvents under reduced pressure afforded Up292 as a crude compound which was purified by column chromatography over silica gel using hexanes: Ethyl acetate mixture (6:4) as eluents to give the title compound Up292 as a yellow solid (0.121 g, 76.5%). ESIMS (m/z): 451 (M+1)

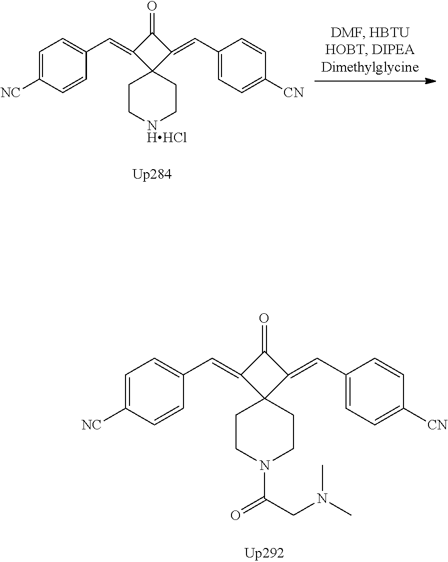

### Example 1: Inhibition of Cancer Cell Proliferation and Colony Formation by Up Therapeutics Compounds (Up compounds)

Treatment of cancer cells with Up compounds inhibited cell proliferation as indicated by MTT assay. Briefly cancer cells incubated with Up compounds for the period of 24 or 48 or 72 hours and the cell viability was measured using MTT assay. IC50 of compounds listed in Table1. Also Up109 and Up117 also significantly blocked colony formation in OV2008 and A2780 cancer cells corresponding to their IC50 values. Both compounds were similarly active in SKOV3 and its taxol resistant line (SKOV3-TR) whereas Taxol show 7-fold difference.

TABLE 1
IC50 values of selected Up Therapeutics compounds (in μM)

| Cell Line | | | | |
|---|---|---|---|---|
| Name | Type | Up101 | Up 109 | Up117 |
| MM.1S | Multiple Myeloma | | 0.035 | 0.042 |
| RPMI8226 | Multiple Myeloma | | 0.123 | 0.14 |
| RPMI8226-R | Multiple Myeloma | | 0.061 | 0.078 |
| LNCaP | Prostate Cancer | | 0.065 | 0.087 |
| PC-3 | Prostate Cancer | | 0.057 | 0.074 |
| DU145 | Prostate Cancer | | 0.077 | 0.13 |
| ES2 | Ovarian Cancer | 0.453 | 0.022 | 0.041 |
| OVCAR3 | Ovarian Cancer | 0.761 | 0.021 | |
| OVCAR5 | Ovarian Cancer | 0.592 | 0.019 | 0.037 |
| SKOV3 | Ovarian Cancer | >1.25 | 0.045 | 0.157 |
| SKOV3-TR | Ovarian Cancer | >1.25 | 0.058 | 0.202 |
| A2780 | Ovarian Cancer | 0.365 | 0.013 | 0.022 |
| OV2008 | Ovarian Cancer | 0.968 | 0.034 | 0.047 |
| HS578T | Triple Negative | | 0.017 | 0.032 |
| | Breast Cancer | | | |
| MDA-MB-231 | Triple Negative | | 0.038 | 0.056 |
| | Breast Cancer | | | |
| HCC1806 | Triple Negative | | 0.042 | 0.071 |
| | Breast Cancer | | | |
| HeLa | Cervical Cancer | | 0.075 | 0.089 |
| HepG2 | Liver Cancer | | 0.118 | 0.447 |
| HFF(primary human | Normal Cell line | >1.25 | >1.25 | >1.25 |
| foreskin fibroblast) | | | | |

### Example 2

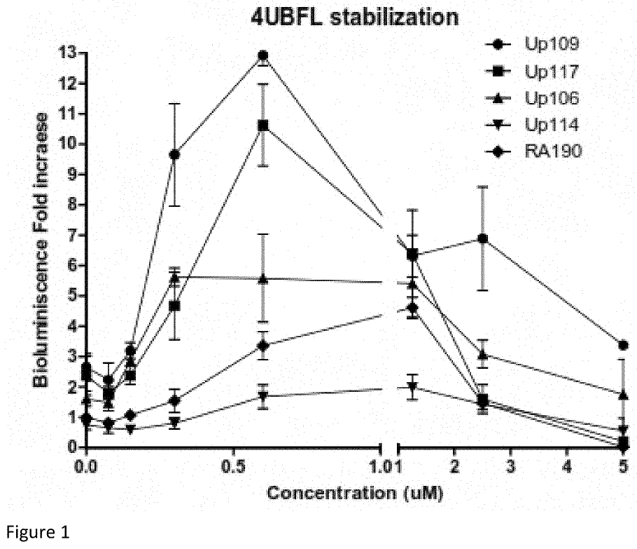

To measure proteasome function in live cells, we utilized an engineered ubiquitin-firefly (4Ub-FL) reporter in which four copies of mutant ubiquitin (ubiquitin G76V) gene are fused to the N-terminus of the firefly luciferase (FL) gene. The results of this experiment, shown in FIG. 1, reveal that the 4Ub-FL reporter protein is rapidly degraded by the proteasome. Importantly, treatment of 293TT cells expressing 4Ub-FL gene with proteasome inhibitors results in its stabilization and an increase in luciferase activity. Interestingly, treatment of Up109 and Up117 produced a greater increase in the 4Ub-FL bioluminescence than RA190 in a dose dependent manner. Up109 shows thirteen-fold increase in bioluminescence whereas eleven and fivefold increase was observed with Up117 and RA190 respectively compared to before treatment.

### Example 3

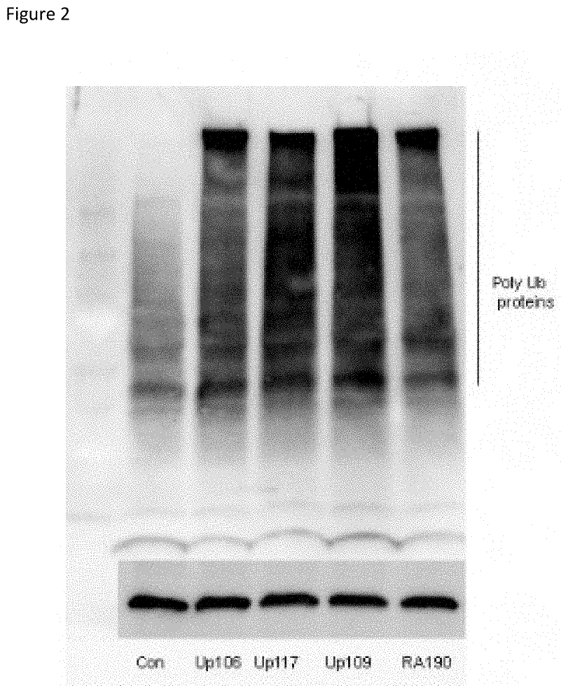

Accumulation of poly ubiquitinated proteins is a general phenomenon of proteasome inhibition. We examined the impact of these compounds on the levels of polyubiquitinated proteins in HeLa cells by anti-K48-linked ubiquitin immunoblot analysis. The results of this experiment, shown in FIG. 2, reveal that treatment of HeLa cells with Up109 or Up117 (4 hr) dramatically increased the levels of K48-linked polyubiquitinated proteins similarly to RA190. However, accumulated K48 polyubiquitinated proteins observed following exposure to compounds exhibited a higher molecular weight than that seen in Bortezomib-treated cells and occurred more rapidly. These results suggest that the toxicity exerted by Up109 and Up117 in cervical cancer cells is associated with a prior accumulation of high-molecular-weight polyubiquitinated proteins and occurs by a mechanism distinct to Bortezomib.

### Example 4

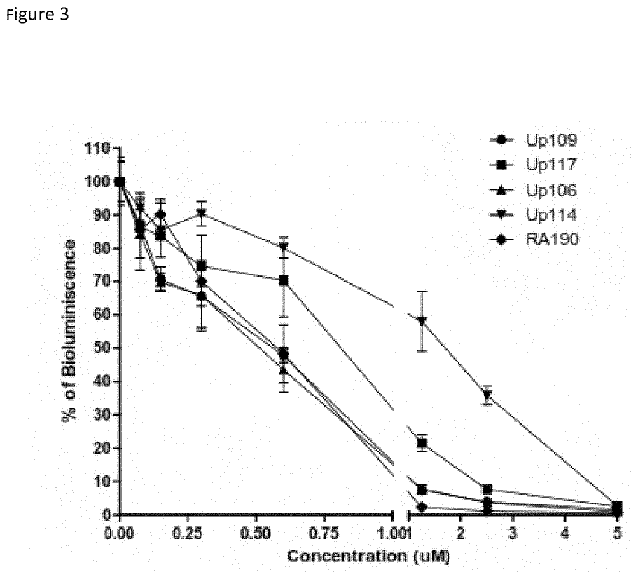

NFKB is constitutively activated in many cancers including high grade cervical intraepithelial neoplasia (CIN) and cervical cancer. Stimulation of 293 cells carrying an NFKB reporter construct with human TNF-a leads to increased reporter activity, and Up109, UP117 and RA190 produced a significant dose-dependent decrease of reporter activity after stimulation with TNF-a (FIG. 3). Stabilization of IkB -a was observed in HeLa cells post treatment with Up109 and Up117 in the presence of TNF-a.

### Example 5

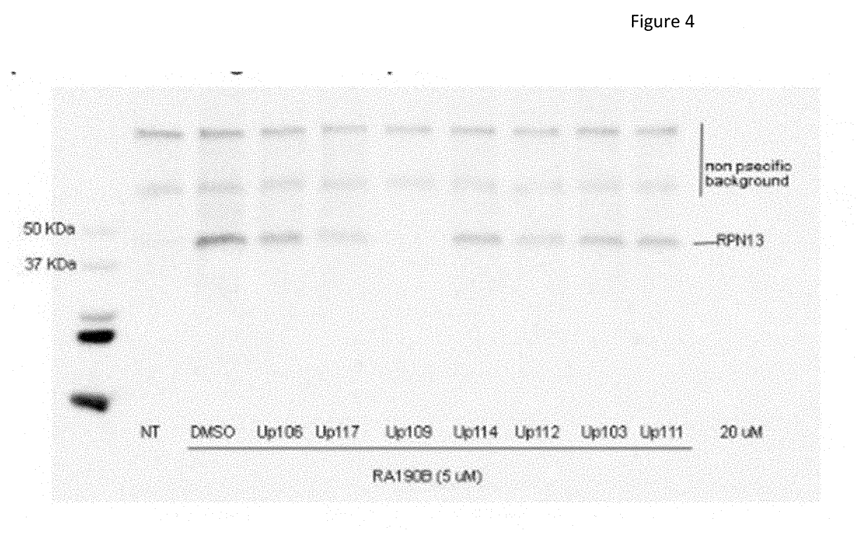

To identify the cellular target, a competition assay was performed using RA190B probe. Earlier we showed that biotinylated RA190 (RA190B) covalently binds to RPN13. We used RA190B as a probe to determine the Up compound binding to RPN13. HeLa cell lysate was pretreated with Up109 or Up117 and then subsequently treated with RA190B. Lysate was denatured under reduced conditions and proteins were separated on a gel and probed with HRPStreptavidin. FIG. 4 shows that disappearance of RA190B labeling of the 42 kDa protein in the presence of Up109 and UP117 indicates competition with RA190B for binding to RPN13.

### Example 6: Up Compounds Accumulated Poly Ubiquinated Proteins In Vitro

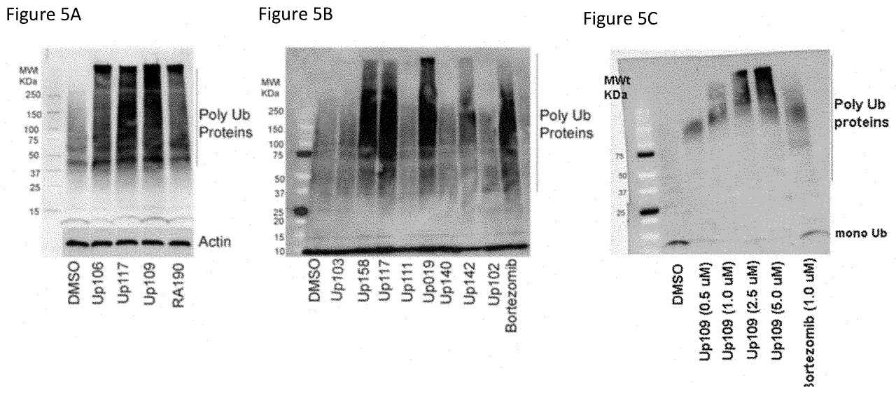

Accumulation of poly ubiquitinated proteins is a general phenomenon of proteasome inhibition. We examined the impact of these compounds on the levels of polyubiquitinated proteins cancer cells by anti-ubiquitin immunoblot analysis. Cancer cell lines (HeLa, OV2008 and LNCaP) treated with Up compounds for the period of indicated time and the cells were lysed and subjected to Westernblot Analysis. Immunoblot with anti-Ubiqutin antibody show the accumulation of polyUbiquitinated proteins in treated cells (FIG. 5). HeLa cells were probed with K48-linked anti-Ub antibody which recognizes the proteins tagged with ubiqiuitin through K-48 linkage. K48-linked polyubiquitin chains attached to substrate proteins often serve as a recognition sequence for targeting and destruction of the substrate by the 26S Proteasome, OV2008 and LNCaP cells were probed with anti-Lib antibody.

### Example 7: Up109 Stabilized Proteasome Dependent Reporter Protein

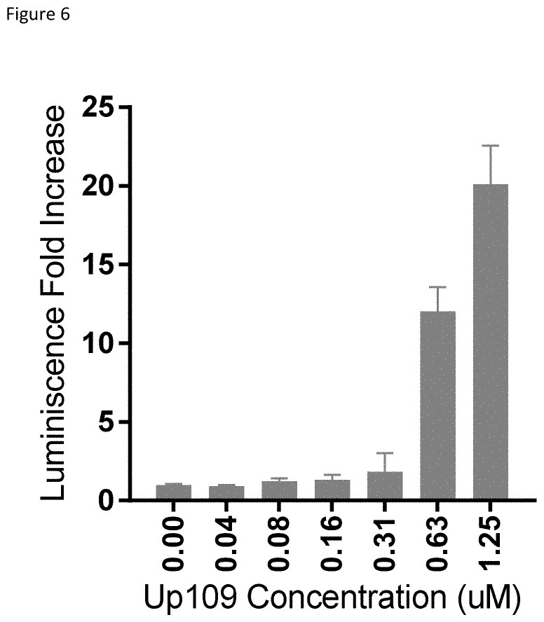

To measure proteasome function in live cells, we utilized an engineered ubiquitin-firefly (4Ub-FL) reporter in which four copies of mutant ubiquitin (ubiquitin G76V) gene are fused to the N-terminus of the firefly luciferase (FL) gene. The 4Ub-FL reporter protein is rapidly degraded by the proteasome (FIG. 6). Importantly, treatment of 293TT cells expressing 4Ub-FL gene with Up109 results in its stabilization and an increase in luciferase activity. Treatment of Up109 produced a greater increase in the 4Ub-FL bioluminescence in a dose dependent manner.

### Example 8: Up109 and Up117 Binds to RPN13

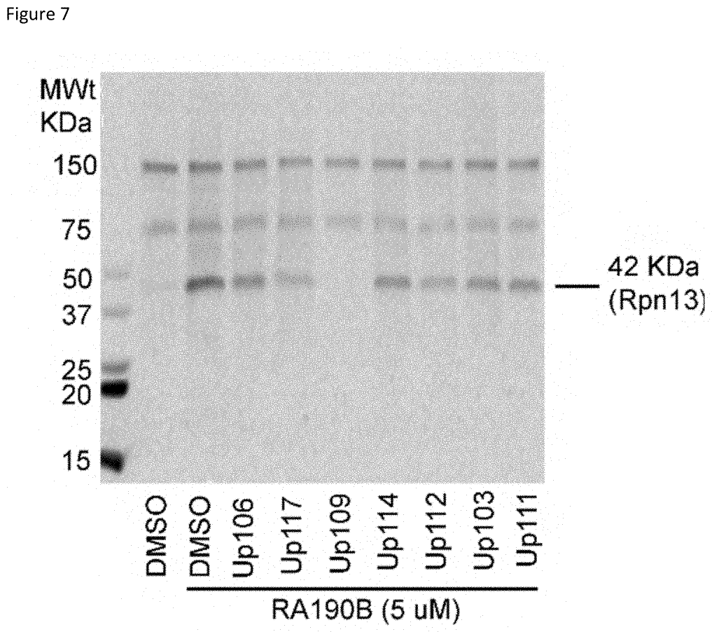

To identify the cellular target, a competition assay was performed using RA190B probe. Earlier we showed that biotinylated RA190 (RA190B) covalently binds to RPN13. We used RA190B as a probe to determine the Up compounds binding to RPN13. OV2008 cell lysate was pretreated with Up compounds (25 μM) and then subsequently treated with RA190B (5 μM). Lysate was denatured under reduced conditions and proteins were separated on a gel and probed with HRP Streptavidin. Disappearance of RA190B labeling of the RPN13 protein in the presence of Up109 and UP117 indicates competition with RA190B for binding to RPN13 (FIG. 7).

### Example 9: Up109 Elevated ER Stress and Apoptosis

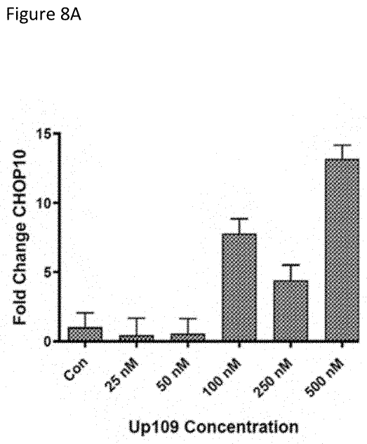

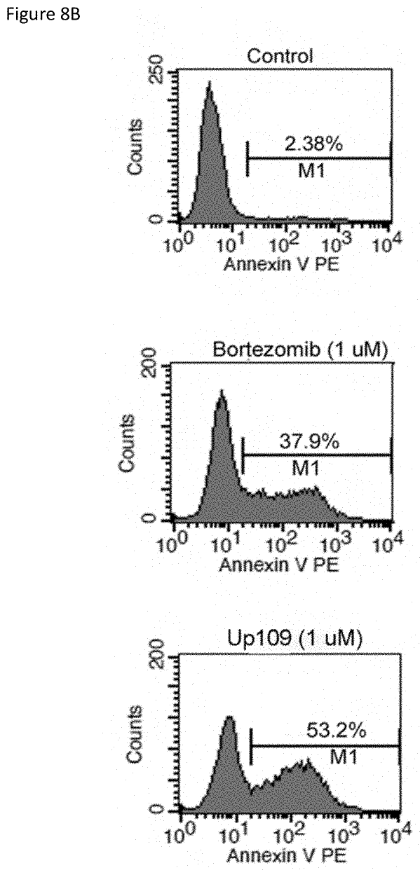

Accumulation of unfolded proteins upon the inhibition of proteasome function rapidly induces endoplasmic reticulum (ER) stress. ER stress triggers an evolutionarily conserved series of signal-transduction events, which constitute the unfolded protein response (UPR). The UPR attempts to restore protein homeostasis by eliminating the accumulated unfolded proteins in the ER; however, if protein homeostasis cannot be restored, apoptosis is triggered. C/EBP homologous protein (CHOP) is elevated at the onset of UPR-induced programmed cell death. Up109 treatment of ES2 cells rapidly up-regulated CHOP-10 mRNA expression (FIG. 8A). Annexin-V positive cells indicate the elevation of apoptosis by Up109 treatment (FIG. 8B).

### Example 10: Up109 Stabilized 4UBFL In Vivo

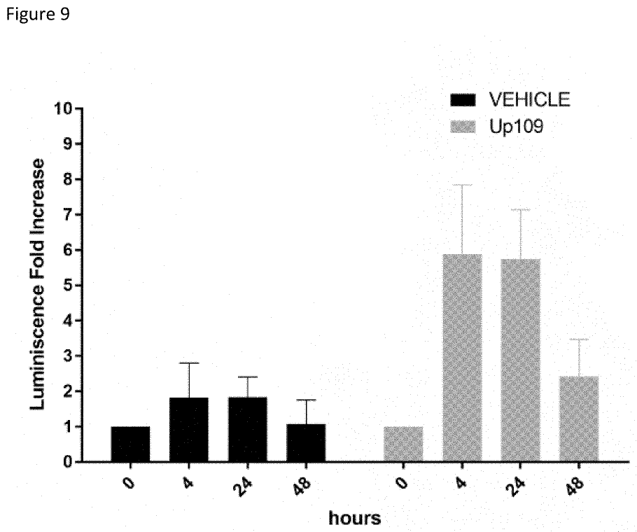

To test for proteasome inhibition by Up109 in vivo, muscle cells of live Balb/c mice transduced with the 4UbFL reporter DNA construct by electroporation. After i.p. injection of luciferin, the enzymic activity of luciferase in the transfected muscle tissue was visualized as bioluminescence using an IVIS imager. At two days post electroporation of the 4UbFL DNA, mice were imaged and base line luminescence recorded. The control group (n=5) of mice was treated i.p. with vehicle alone and another group (n=5) treated i.p with Up109 (40 mg/Kg). After 4 h, 24 h and 48 h post treatment mice were again imaged and luminescence was quantified (FIG. 9). This result indicates (1) Up109 has good accessibility to solid tissue; (2) Up109 could potentially be dosed every other day.

### Example 11: Dose Limiting Toxicity Studies

To identify the optimal dose that induces a clinical effect and operates with acceptable toxicity a set of experiments has been performed on female Balb/C strain mice with Up109. Individual groups of mice (n=3) were injected i.p. with increasing single doses of Up109 (3, 10, 20, 40 and 100 mg/Kg) and the endpoint evaluations included clinical observations and body weights. No clinically apparent sign of adverse effect was found with Up109. Another experiment was done with repeated i.p. doses of Up109 (40 mg/Kg, n=5) every other day for two weeks. No observable toxicities/weight loss were found.

### Example 12: In Vivo Efficacy of Up109 Against ES2 Ovarian Tumor Growth

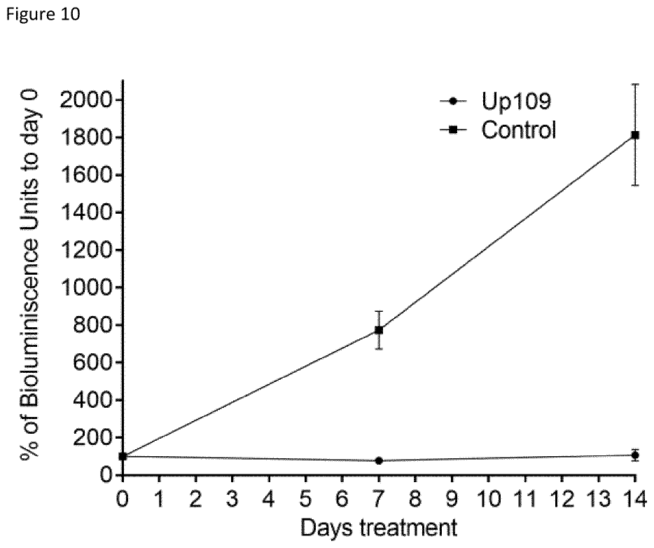

To test the efficacy of Up109 to treat the human ovarian cancer ES2 xenograft model, the luciferase-expressing ES2 cells (ES2-Lu) were inoculated into peritoneal cavity of nude female mice (i.e. orthotopically). Two days after inoculation the mice were imaged for their basal luminescence activity and then randomized into two groups (n=10). First group was treated with the vehicle and the second group was treated with Up109 (10 mg/Kg) on alternate days for two weeks. Mice were imaged after the first and second week of treatment for their luciferase activity. As indicated in FIG. 6, Up109 significantly reduced the tumor burden compared to the vehicle and there was no weight loss and any observable side effects. Tumor burden in the vehicle group mice was excessive and sacrificed at the end of the treatment (FIG. 10).

### Example 13

UpTx Compounds Structural Features and Their Activity Against Cancer Cell Growth

Compounds were screened for their cytotoxic effect against the growth of two cancer cell lines HeLa and SKOV3 for the period of 72 hrs as triplicates and the cell viability was determined using MTT assay. It is surprising that only the compounds possessing strong electron withdrawing groups (—NO2 and —CN substituents) on the aromatic ring show significant increase in the potency compared to other compounds. Data are shown in Table 1 (below).

TABLE 1
UpTx compounds structural features and their activity against cancer cell growth

| | | | Ring | | |
|---|---|---|---|---|---|
| Compound | A2/A4 | R3 | Size | HeLa | SKOV3 |
| Up101 | 4NO2-Phenyl | Boc | [3.5] | >1.25 | >1.25 |
| Up102 | 4Cl-Phenyl | Acetyl | [3.5] | >1.25 | >1.25 |
| Up103 | 3,4diCl-Phenyl | Acetyl | [3.5] | >1.25 | >1.25 |
| Up104 | 4NO2-Phenyl | H | [3.5] | 0.18 | 0.35 |
| Up106 | 4NO2-Phenyl | Phenylalanine(as | [3.5] | 0.134 | 0.193 |
| | | amide) | | | |
| Up108 | 3,4-diCl-Phenyl | H | [3.5] | >1.25 | >1.25 |
| Up109 | 4NO2-Phenyl | Acryloyl | [3.5] | 0.045 | 0.052 |
| Up110 | 3,4-diCl-Phenyl | H | [3.3] | >1.25 | >1.25 |
| Up111 | 3,4-diCl-Phenyl | Acryloyl | [3.3] | >1.25 | >1.25 |
| Up112 | 3,4-diCl-Phenyl | Acryloyl | [3.5] | >1.25 | >1.25 |
| Up114 | 4Cl-Phenyl | Acryloyl | [3.5] | >1.25 | >1.25 |
| Up117 | 4NO2-Phenyl | Chloroacetamide- | [3.5] | 0.059 | 0.157 |
| | | phenyalanine (as | | | |
| | | amide) | | | |
| Up131 | 4F-Phenyl | H | [3.5] | >1.25 | >1.25 |
| Up132 | 4F-Phenyl | Acryloyl | [3.5] | >1.25 | >1.25 |
| Up134 | 2F-Phenyl | H | [3.5] | >1.25 | >1.25 |
| Up135 | 2F-Phenyl | Acryloyl | [3.5] | >1.25 | >1.25 |
| Up137 | Cinnamyl | H | [3.5] | >1.25 | >1.25 |
| Up140 | Phenyl | H | [3.5] | >1.25 | >1.25 |
| Up142 | Phenyl | Acryloyl | [3.5] | >1.25 | >1.25 |
| Up144 | Pyridine | H | [3.5] | >1.25 | >1.25 |
| Up148 | Thiazole | H | [3.5] | >1.25 | >1.25 |
| Up158 | 4NO2-Phenyl | Acetyl | [3.5] | 0.152 | 0.276 |
| Up161 | 4NO2-Phenyl | Serine (as amide) | [3.5] | >1.25 | >1.25 |
| Up169 | 4Cl-Phenyl | H | [3.4] | >1.25 | >1.25 |
| Up171 | 4Cl-Phenyl | Acryloyl | [3.4] | >1.25 | >1.25 |
| Up173 | 4NO2-Phenyl | H | [3.4] | 0.16 | 0.29 |
| Up188 | 4NO2-Phenyl | SO2Ph | [3.5] | >1.25 | >1.25 |
| Up198 | 4Br-Phenyl | H | [3.5] | >1.25 | >1.25 |
| Up199 | 4Br-Phenyl | Acryloyl | [3.5] | >1.25 | >1.25 |
| Up200 | 4NO2-Phenyl | Probenecid | [3.5] | >1.2 | >1.25 |
| Up201 | 4Br-Phenyl | methylNapthylene | [3.5] | >1.25 | >1.25 |
| Up284 | 4CN-Phenyl | H | [3.5] | 0.04 | 0.071 |
| Up285 | 4CN-Phenyl | Acryloyl | [3.5] | 0.025 | 0.06 |
| Up288 | 4CN-Phenyl | benzoyl | [3.5] | 0.28 | 0.39 |
| Up290 | 4CN-Phenyl | CO—CF3 | [3.5] | 0.15 | 0.29 |
| Up291 | 4CN-Phenyl | Valproic | [3.5] | 0.25 | 0.42 |
| Up292 | 4CN-Phenyl | N,N-Dimethylglycine (as | [3.5] | 0.23 | 0.35 |
| | | amide) | | | |
| Up302 | 4CN-Phenyl | H | [3.4] | 0.08 | 0.17 |
| Up306 | 4CN-Phenyl | Acryloyl | [3.4] | 0.05 | 0.12 |
| Up310 | 4CN-Phenyl | CH2—C≡C— | [3.5] | 0.5 | 0.75 |

### Example 14

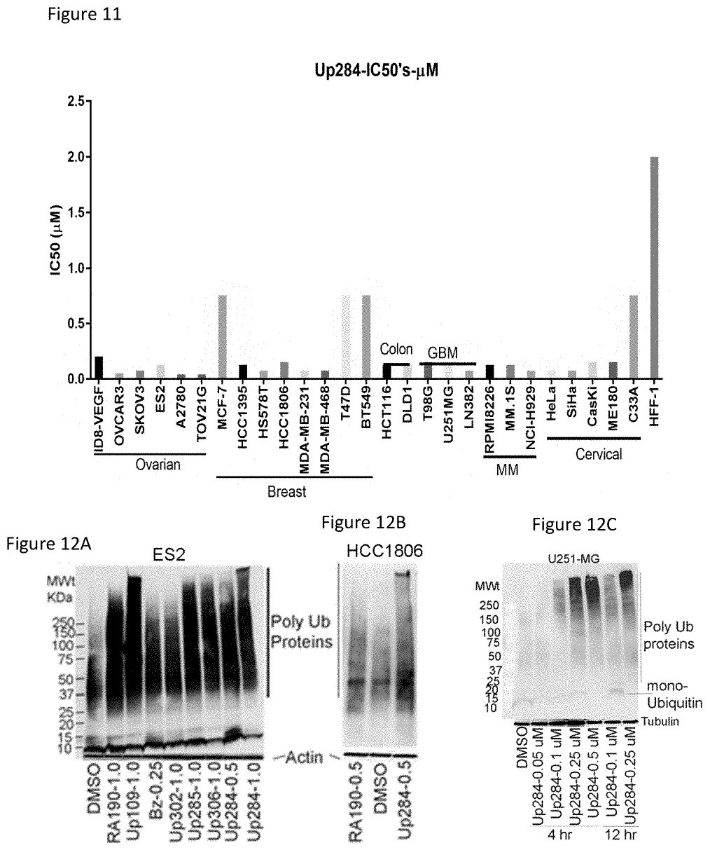

Effect of UP284 against the viability of different cancer cell lines treated with Up284 for 72 hours. Cancer cell lines were plated in a 96 well plate as triplicates at the density of 2000-10000 cells/well in 100 μL growth medium. After 18-24 hours cells were treated with Up284 for the period of 72 hrs and the cell viability was assessed colorimetrically at 570 nm using MTT reagent (Thiazolyl Blue Tetrazolium Bromide, Sigma, M5655) according to the manufacturer protocol. Results are shown in FIG. 11.

### Example 15

Evidence of proteasome inhibition. Accumulation of poly ubiquitinated proteins is a general phenomenon of proteasome inhibition. We examined the impact of these compounds on the levels of polyubiquitinated proteins in canceU251-MG) treated with Up compounds for the period of indicated times and the cells were lysed and subjected to Western blot Analysis. Immunoblot with anti-Ubiqutin antibody show the accumulation of polyUbiquitinated proteins in treated cells. FIG. 12 shows the results of this experiment. FIGS. 12(A) ES2; 12(B) HCCl806; and 12 (C) U251-MG cells showing accumulation of high MW polyUb proteins with Up compounds treatment at indicated times and concentrations (in μM) in a Western blot using ubiquitin antibody. Bz=bortezomib. Actin or Tubulin used as positive controls.

### Example 16

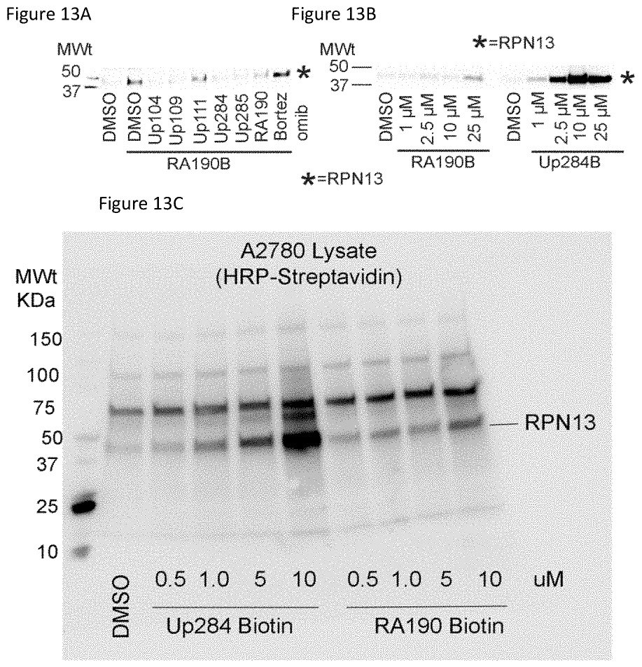

Up284 binds to RPN13 much more strongly than RA190. To identify the cellular target, a competition assay was performed using RA190B probe. Earlier we showed that biotinylated RA190 (RA190B) covalently binds to RPN13. We used RA190B as a probe to determine the Up compounds binding to RPN13. ES2 cell lysate was pretreated with Up compounds (5 μM) and then subsequently treated with RA190B (20 μM). Lysate was denatured under reduced conditions and proteins were separated on a gel and probed with HRP Streptavidin. Disappearance of RA190B labeling of the RPN13 protein in the presence of compounds indicates competition with RA190B for binding to RPN13 (A). In a similar way ES2 cell lysate (B) and A2780 cell lysate (C) were directly labelled with Up284B and RA190B. impact of these compounds. Results are shown in FIG. 13.

FIG. 13A shows the results from an experiment where ES2 cell lysate pretreated with Up compounds (5 μM) and then labelled with biotinylated RA190 (RA190B 20 μM). The lysate was separated by SDS-PAGE, transferred to a membrane and probed with HRP-streptavidin. Up compounds, but not Bz, compete RA190B binding to 42 KDa RPN13. FIG. 13(B) shows the results from an experiment where ES2 cell lysate treated with RA190B and biotinylated Up284 (Up284B) at indicated concentrations and probed for HRP-Streptavidin. FIG. 13(C) shows the results from an experiment where A2780 cell lysate treated with Up284biotin and RA190 biotin at indicated concentrations. The lysate was separated by SDS-PAGE, transferred to a membrane and probed with HRP-streptavidin.

### Example 17

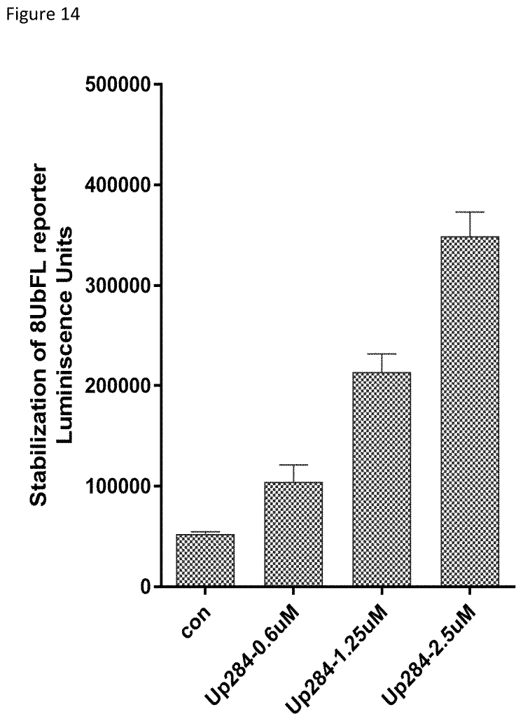

Up284 stabilized proteasome dependent reporter protein. To measure proteasome function in live cells, we utilized an engineered ubiquitin-firefly (8Ub-FL) reporter in which eight copies of mutant ubiquitin (ubiquitin G76V) gene are fused to the N-terminus of the firefly luciferase (FL) gene. The 8Ub-FL reporter protein is rapidly degraded by the proteasome. Importantly, treatment of 293TT cells expressing 8Ub-FL gene with Up284 results in its stabilization and an increase in luciferase activity. Treatment of Up284 produced a greater increase in the 8Ub-FL bioluminescence in a dose dependent manner (FIG. 14).

### Example 18

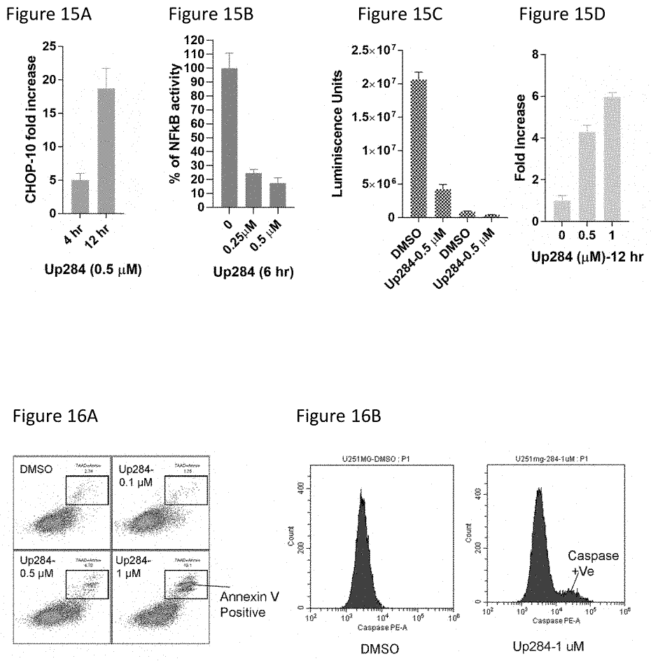

Up284 induced ER stress, Inhibited NF-kb activity, reduced GSH/GSSG levels and increased ROS in glioblastoma cell line U251MG: FIG. 15(A) RT-qPCR of U251MG cells showing ER stress response element CHOP-10 increase with Up284 treatment. FIG. 15(B) Bioluminescence of 293 cells expressing NFkB dependent luciferase show reduced NFkB activity with Up284 treatment. with indicated doses of Up284. FIG. 15(C) U251MG cells showing reduced GSH/GSSG activities with Up284 treatment (G) increased ROS is seen with Up284 treatment.

### Example 19

Up284 treatment caused apoptotic cell death in glioblastoma cell line U251MG: FIG. 16(A): FACS analysis of U251MG cells showing Annexin-V positive cells with after 12-hour Up284 treatment. FIG. 16(B) FACS analysis showing cleaved caspase after 12 hr Up284 treatment.

1. Compounds having the structure of formula either I, or II, or III, 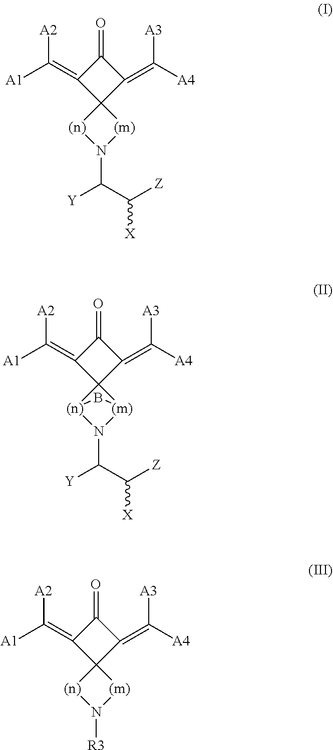
or a pharmaceutically acceptable salt thereof
where in each of A1, A2, A3, and A4 is one of:
(i) phenyl, optionally substituted with 1-5 substituents selected from the group consisting of R1, OR1, NR1R2, S(O)qR1, SO₂NR1R2, NR1SO₂R2, C(O)R1, C(O)OR1, C(O)NR1R2, NR1C(O)R2, NR1C(O)OR2, CF₃, and OCF₃;
(ii) naphthyl, optionally substituted with 1-5 substituents selected from the consisting of R1, OR1, NR1R2, S(O)qR1, SO₂NR1R2, NR1SO₂R2, C(O)R1, C(O)OR1, C(O)NR1R2, NR1C(O)R2, NR1C(O)OR2, CF₃, and OCF₃;
(iii) a 5 or 6 membered monocyclic heteroaryl group, having 1-3 heteroatoms selected from the group consisting of 0, N, and S, optionally substituted with 1-3 substituents selected from the group consisting of R1, OR1, NR1R2, S(O)qR1, SO₂NR1R2, NR1SO₂R2, C(O)R1, C(O)OR1, C(O)NR1R2, NR1C(O)R2, NR1C(O)OR2, CF₃, and OCF₃; and
(iv) an 8 to 10 membered bicyclic heteroalkyl group containing 1-3 heteroatoms selected from the group consisting of O, N, and S; and the second ring is fused to the first ring using 3 to 4 carbon atoms, and the bicyclic hetero aryl group is optionally substituted with 1-3 substituents selected from the group consisting of R1, OR1, NR1R2, S(O)qR1, SO₂NR1R2, NR1SO₂R2, C(O)R1, C(O)OR1, C(O)NR1 R2, NR1 C(O)R2, NR1C(O)OR2, CF3, and OCF3;
(v) any group belongs to R1 or R2 wherein n represents number of atoms ranging from 0-4 (0,1,2,3,4) and can be C, N, or O, wherein when n is N, it can be NH, NR1 or NR2;
wherein X is Hydrogen, OR1 or NP, wherein P is selected from the group consisting of R1, C(O)R1, C(O)OR1, C(O)NR1 R2, S—N(R1)COOR1, and S—N(R1), wherein Y is selected from the group consisting of O, S, NR1 and CR1 R2, and wherein R1 and R2 are selected from the group consisting of —H, —NO2, —OH, —COON, —NH2, halogen, —CN and C1-C14 linear or branched alkyl groups, that are optionally substituted with 1-3 substituents selected from the group consisting of C1-C4 linear or branched alkyl, up to perhalo substituted C1-C14 linear or branched alkyl, C1-C4 alkoxy, hydrogen, nitro, hydroxyl, carboxy, amino, C1-C14 alkylamino, C-i-C-n dialkylamino, halogen, and cyano;
wherein Z is selected from the group consisting of hydrogen; C1 to C14 linear, branched, or cyclic alkyls; alkenyls, phenyl; benzyl, 1-5 substituted benzyl, C1 to C3 alkyl-phenyl, wherein the alkyl moiety is optionally substituted with halogen up to perhalo; up to perhalo substituted C1 to C14 linear or branched alkyls; —(CH2)q-K, where K is a 5 or 6 membered monocyclic heterocyclic ring, containing 1 to 4 atoms selected from oxygen, nitrogen and sulfur, which is saturated, partially saturated, or aromatic, or an 8 to 10 membered bicyclic heteroaryl having 1-4 heteroatoms selected from the group consisting of O, N and S, wherein said alkyl moiety is optionally substituted with halogen up to perhalo, and wherein the variable q is an integer ranging from 0 to 4;
wherein B is (i) R1, C(O)R1, C(O)OR1, C(O)NR1R2, S—N(R)COOR1, S—N(R1)COO(B), S(B); and
wherein each R1-R2, other than per-halo substituted C1-C14 linear or branched alkyl, is optionally substituted with 1-3 substituents independently selected from the group consisting of C-1-C14 linear or branched alkyl, up to perhalo substituted C1-C14 linear or branched alkyl, C1-C3 alkoxy, hydroxyl, carboxy, amino, C1-C3 alkylamino, C1-C6 dialkylamino, halogen, cyano; and
wherein R3 is H, C1-6-alkyl, C2-6-alkenyl; C1-3-alkoxy-C1-6-alkyl-; C1-3-alkoxy-C2-6-alkenyl-;aryl-C0-6-alkyl-; heteroaryl-C0-6-alkyl-; heterocyclyl-C0-6-alkyl-; cycloalkyl-C0-6-alkyl-; [symbol] C1-6-alkyl-COOC1-6-alkyl; [symbol] C2-6-alkyl-aryloxy; C1-6-alkyl-heteroaryl; C1-6-alkyl-heterocyclyl; C1-6-alkyl-cycloalkyl; C1-6-alkyl-aryl; COR⁴, wherein R⁴ is selected from: C1-6-alkyl; C2-6-alkenyl; C1-6-alkoxy; C1-3-alkoxy-C1-6-alkyl-; C1-3-alkoxy-C2-6-alkenyl-; aryl-C0-6-alkyl-; heteroaryl-C0-6-alkyl-;heterocyclyl-C0-6-alkyl-; cycloalkyl-C0-6-alkyl-; —C1-6-alkyl-COOC1-6-alkyl; NH₂; —NHC 1-6-alkyl; —N(C 1-6-alkyl)₂; —C0-6-alkyl-aryloxy.

2. A compound according to claim 1 which binds to proteasomal proteins as proteasome inhibitor.

3. The compound according to claim 1, wherein the compound has the formula

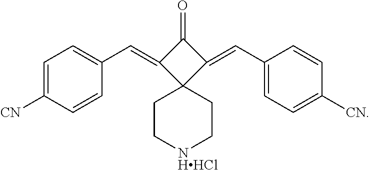

4. A method of inhibiting proteasomes in a mammal by administering to the mammal an effective amount of the compound of claim 1.

5. A method of treating or a disease in a mammal by administering to the mammal a therapeutically effective dose of the compound of claim 1.

6. The method of claim 5 wherein the mammal is a human, or a dog, or a cat.

7. The method of claim 5 wherein the disease is a type of cancer or diabetes or neurological disorders.

8. The method of claim 5 wherein the compound of is administered alone or in combination with at least one other therapeutic agent or radiation.
